(19) United States
(12) Patent Application Publication
(10) Pub. No.: US 2024/0285693 A1
(43) Pub. Date: Aug. 29, 2024

(54) EXPANSION OF RETINAL PIGMENT EPITHELIUM CELLS

(71) Applicant: Cell Cure Neurosciences Ltd., Jerusalem (IL)

(72) Inventors: Dana Hayoun Neeman, Jerusalem (IL); Ofer Wiser, Jerusalem (IL); Ravid Tikotzki, Jerusalem (IL); Lilach Alon, Jerusalem (IL)

(21) Appl. No.: 18/424,000

(22) Filed: Jan. 26, 2024

Related U.S. Application Data
(63) Continuation of application No. PCT/US2022/038594, filed on Jul. 27, 2022.
(60) Provisional application No. 63/226,741, filed on Jul. 28, 2021.

(51) Int. Cl. A61K 35/30; C12N 5/079

(52) U.S. Cl. CPC A61K 35/30; C12N 5/0621; C12N 2501/15; C12N 2506/02; C12N 2531/00

(57) ABSTRACT

Presented herein are methods and compositions for expanding RPE cells with the use of a suspendable cell support matrix. Also provided are pharmaceutical compositions containing RPE cells, and methods of treating an eye disorder or disease using RPE cells.

### RELATED APPLICATIONS

This application claims the benefit of priority to U.S. Provisional Application No. 63/226,741, filed Jul. 28, 2021, the entire contents of which are incorporated herein by reference in their entirety.

### BACKGROUND

The retinal pigment epithelium (RPE) is a monolayer of neuroepithelium-derived pigmented cells that lays on a Bruch's membrane between the photoreceptor outer segments (POS) and the choroidal vasculature. The RPE monolayer is critical to the function and health of the photoreceptors. Dysfunction, injury, and loss of retinal pigment epithelium (RPE) cells are prominent features of certain eye diseases and disorders, such as age-related macular degeneration (AMD), hereditary macular degenerations including Best disease (the early onset form of vitelliform macular dystrophy), and subtypes of retinitis pigmentosa (RP). The transplantation of RPE (and photoreceptors) into the retina of those affected with such diseases can be used as cell replacement therapy in retinal diseases where RPE have degenerated.

Human fetal and adult RPE have been used as a donor source for allogeneic transplantation. However, practical problems in obtaining sufficient tissue supply and the ethical concerns regarding the use of tissues from aborted fetuses limit widespread use of these donor sources. Given the limitations in the supply of adult and fetal RPE grafts, the potential of alternative donor sources has been studied.

Human pluripotent stem cells provide significant advantages as a source of RPE cells for transplantation. Their pluripotent developmental potential enables their differentiation into authentic functional RPE cells, and given their potential for infinite self-renewal, they can serve as an unlimited donor source of RPE cells. Indeed, it has been demonstrated that human embryonic stem cells (hESCs) and human induced pluripotent stem cells (iPSCs) may differentiate into RPE cells in vitro, attenuate retinal degeneration and preserve visual function after sub retinal implantation. Therefore, hESCs can be an unlimited source for the production of RPE cells for cell therapy.

However, the number of patients requiring treatment using RPE cells is projected to exceed 200 million worldwide. These vast numbers pose a manufacturing challenge, as the required industrial allogenic lot size must be increased to many billions of RPE cells. Manufacturing platforms for anchorage-dependent cell types such as RPE cells traditionally employ two-dimensional culture methods. These platforms are not optimal for industrial manufacturing, as they are labor intensive, have very large foot prints, and consume an excess of resources. Being uncontrolled open systems, they also harbor high risk for contamination and variability between lots.

The present disclosure addresses these and other shortcomings in the field of regenerative medicine and RPE cell therapy.

### BRIEF SUMMARY OF THE INVENTION

Described herein are methods for the expansion of retinal pigment epithelial (RPE) cells, pharmaceutical compositions of RPE cells, and methods for the treatment of a disorder of the eye with pharmaceutical compositions generated using said methods. In embodiments, the disease is age-related macular degeneration (AMD). In embodiments, the disease is hereditary macular degenerations including Best disease (the early onset form of vitelliform macular dystrophy), or a subtype of retinitis pigmentosa (RP).

In an aspect, provided herein, is a method for the expansion of retinal pigment epithelial (RPE) cells, the method includes: providing a population of RPE cells, wherein the population of RPE cells was differentiated from pluripotent stem cells; inoculating a medium comprising a first suspendable cell support matrix, such as a microcarrier, with the population of RPE cells; and expanding the population of RPE cells on the first suspendable cell support matrix in dynamic suspension to provide an expanded population of RPE cells.

In an aspect, provided herein, is a pharmaceutical composition containing RPE cells generated by the method for the expansion of retinal pigment epithelial (RPE) cells, the method including: providing a population of RPE cells, wherein the population of RPE cells was differentiated from pluripotent stem cells; inoculating a medium comprising a first suspendable cell support matrix with the population of RPE cells; and expanding the population of RPE cells on the first suspendable cell support matrix in dynamic suspension to provide an expanded population of RPE cells.

In an aspect, provided herein, is a method of treating a disorder or disease of the eye, the method including transplanting into the retinal tissue of a patient in need thereof a pharmaceutical composition containing RPE cells generated by the method for the expansion of retinal pigment epithelial (RPE) cells, the method including: providing a population of RPE cells, wherein the population of RPE cells was differentiated from pluripotent stem cells; inoculating a medium comprising a first suspendable cell support matrix with the population of RPE cells; and expanding the population of RPE cells on the first suspendable cell support matrix in dynamic suspension to provide an expanded population of RPE cells.

### BRIEF DESCRIPTION OF THE DRAWINGS

The technology described herein will be more fully understood by reference to the following drawings which are for illustrative purposes only:

FIG. 1 is a series of images of RPE cells during passage 1 (P1) of RPE expansion. Magnification is 10×. The images show (from left to right): day 4 post seeding, day 7 post seeding, day 8 post seeding, and day 9 post seeding (harvest day) RPE cells. The RPE cells start to show a polygonal monolayer membrane at day 4 post seeding, and reach high density polygonal morphology on day 9 (harvesting day).

FIG. 2 shows morphological assessment of indicated MCS studies showing the outer layer of RPE cells on microcarriers' (MC) surface. Representative phase images (4× objective) of RPE cells attached to MC near the end of passage (day number is indicated).

FIG. 3 is a graph showing a comparison of percent dissolved oxygen (% DO) trends during multiple RPE cells expansion runs in the Single Use Bioreactor (SUB) over the course of about 10-12 days. The third fed-batch of MCS 14 started on Day 10, four days pre-harvesting. (Due to technical reasons, MCS 14 data up to Day 6 was lost.)

FIG. 4 is a series of images showing MC-cell population density evolvement throughout study MCS 11B. Though inoculation cell density of 120×10³ cells/cm² is targeted, cells do not evenly disperse on the MC. This uneven dispersal leads to a fraction of the MC being eventually more densely populated, resulting in more mature RPE cells that populate those MC over time. Arrows point to heterogeneously populated MC in samples taken on different days. Variations in MC-cell densities are apparent as early as Day 4 post inoculation. From left to right, Day 4, Day 4 (enlarged), Day 14, and Day 14.

FIG. 5 is a flowchart illustrating an embodiment of large scale RPE cell production process.

FIG. 6 is a microscopic image of PMEL17 and DAPI stained microcarrier particles coated with RPE cells.

FIG. 7 shows the growth of RPE cell monolayers on microcarriers (top row) vs. on a T175 flask (bottom row). Microscopic images were taken at day 3, 7 and 14 after inoculating each culture.

FIG. 8 is a schematic of an example method of making an intermediate cell bank (ICB) from the differentiated RPE cells. Cells are harvested and frozen in aliquots at the end of differentiation (P0), as indicated. The cells from the ICB can be used to inoculate microcarriers in large-scale bioreactors for expansion of the RPE cells.

FIG. 9 shows two examples of schematics for production of RPE cells. The top schematic shows the RPE cell expansion phase without use of an intermediate cell bank (ICB). The bottom schematic shows the RPE cell expansion phase with the use of the intermediate cell bank (ICB).

FIG. 10 is a flowchart illustrating examples of methods of differentiation expansion for large scale OPREGEN® cell production process, including the use of microcarriers (MC) and an intermediate cell bank (ICB).

FIG. 11 is a flowchart illustrating an example method for differentiating and expanding RPE cells from hESC.

FIG. 12 is a flowchart illustrating example RPE cell expansion methods with the use of an intermediate cell bank (ICB).

FIGS. 13A and 13B show example RPE cell potency assay results. FIG. 13A shows a microscopic image of a high potency mature RPE sample vs. a low potency mature RPE sample. The high potency mature RPE cells exhibit a confluent, uniform, polygonal monolayer morphology. The low potency mature RPE cells exhibit a sub-confluent, non-uniform morphology with holes. FIG. 13B, left panel, shows a bar graph of a plot of transepithelial resistance (TEER, in Ω*cm²) at day 14 for (1) high potency RPE cells, and (2) low potency RPE cells. The bar graph in the right panel shows the PEDF:VEGF polarized secretion ratio for (1) high potency mature RPE cells and (2) low potency mature RPE cells.

FIG. 14 shows the scatter plots of a flow cytometry (FCM) analysis of the different stages of RPE cell expansion and maturation, where cells are dual-labeled with CRALBP_FITC_488 and PMEL_AlexaFlour_647. The top left scatter plot shows the cell population during hESC expansion. The top right scatter plot shows the cell population at the end of differentiation. The bottom left scatter plot shows the cells during RPE expansion. The bottom right scatter plot shows the cells at the end of the RPE expansion and maturation process. The cells at the end of the expansion/maturation process are >95% RPE cells.

FIG. 15 shows, from left to right, a bar graph of RPE purity/identity biomarker expression (CRALBP/PMEL17) during OPREGEN® production, a bar graph of RPE maturation biomarker (PEDF) secretion during OPREGEN® production, and the mature RPE cell morphology at the end of the differentiation, expansion, and maturation process.

FIGS. 16A and 16B show EXP27A Preliminary screening study of the indicated 6 types of microcarriers for 1 day to evaluate RPE adherence. FIG. 16A are representative phase images of RPE cells attached to all MC types in the presence of 20% HS. FIG. 16B is a graph showing the % of adherence to each MC type in all the tested HS concentrations at 24h, as calculated by the % of harvested cells out of the total seeded cells per well. (Bars L to R in each set: 20% HS; 5% HS; 0.5% HS; 0% HS)

FIG. 17 are images showing EXP27A preliminary screening study of the indicated 6 types of microcarriers for 7 days to evaluate RPE adherence and expansion. The images depict that the cells reached confluency and polytonality in all MC types.

FIG. 18 is a bar graph showing that the highest yield achieved by seeding the RPE cells on the Star-Plus MC. (Bars L to R in each set: 20% HS; 5% HS; 0.5% HS; 0% HS)

FIG. 19A is a bar graph showing total cells calculated for spinner flasks seeded with the indicated cell densities.

FIG. 19B is a graph showing the cell yield calculated for spinner flasks seeded with the indicated cell densities.

FIGS. 20A and 20B are data comparing two feeding regimens along RPE cells expansion in spinner flasks. P1 Spinner flask (fed by 1 media exchange) and T175 flask were harvested and seeded for P2 in either Control T-flask or in spinner flasks. Two spinner flask that were originated from the P1 spinner flask were used to test the 2 feeding regimens, Fed batch and ½ media exchange. The spinner flask that receive the fed batch had higher yield (FIG. 20A). Control P1 spinner flask (EXP 27E MC) and the two P2 spinner flasks (EXP27F MC 1A/2A) showed similar post thaw purity (% CRALBP/PMEL17) values in flow cytometry test (FIG. 20B).

FIGS. 21A and 21B are graphs showing a results summary of studies in which feeding regimen was 12 media exchange vs. studies using fed batch. Analysis show yield (FIG. 21A) and total cells harvested per square cm at end of passage (FIG. 21B).

FIG. 22 are images showing growth of RPE cells on various microcarriers at 3, 10 and 12 days.

### DETAILED DESCRIPTION

After reading this description it will become apparent to one skilled in the art how to implement the invention in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present invention as set forth below.

Before the present invention is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description of the invention is divided into various sections only for the reader's convenience and disclosure found in any section may be combined with that in another section. Titles or subtitles may be used in the specification for the convenience of a reader, which are not intended to influence the scope of the present invention.

To meet the needs of a rapidly expanding patient population, what is needed is large scale production of retinal pigment epithelium (RPE) cells. It has been demonstrated that human embryonic stem cells (hESCs) or human induced pluripotent stem cells (hiPSCs) can be consistently differentiated into functional RPE cells in vitro. The use of such RPE cells in patients with age-related macular degeneration (AMD) in clinical trials shows promising functional recovery. Disclosed herein is a platform for RPE expansion using a suspendable cell support matrix, or a microcarrier (MC). The properties of MC, such as being suspended in solution while providing a surface on which adherent cells can grow, makes them ideal for growing adherent cell culture in closed and controlled environments. Another benefit of using MC for large-scale production is the surface area to volume ratio, which is greatly increased over traditional static culture processes. Thus, cell density can be increased while the required footprint is reduced.

Herein are described methods of using the potential of RPE cells to grow on a suspendable cell support matrix (e.g., without limitation, Star-Plus Microcarriers by SOLOHILL®) for expansion of hESC derived RPE in a large scale closed and controlled environment. For example, in the closed system described herein, differentiated RPE cells may be inoculated within a single bioreactor containing a suspendable cell support matrix that is screened for optimal RPE yield and quality. Cells' oxygen consumption may be automatically monitored and controlled, as can the pH, metabolites, and temperature. The feeding regimen can be done in fed batch mode, in which fresh media and glucose are added as needed. All manipulation, including the suspendable cell support matrix and media addition, cell sampling, harvesting, and filtration, may be done in a controlled and closed environment, using tube welding of single use bags, until the final product of cell suspension in cryomedium is automatically dispensed into cryovials. Controlled large scale freezing of thousands of vials, up to approximately 2300 vials per one hour freezing session, can be achieved.

### Definitions

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing. “Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a composition to accomplish a stated purpose relative to the absence of the composition (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug (e.g., the cells described herein) is an amount of the drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any composition described herein, the therapeutically effective amount can be initially determined from cell culture assays. Target concentrations will be those concentrations of active composition(s) (e.g., cell concentration or number) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring effectiveness of a composition and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the composition being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the composition. Thereafter, the dosage is increased by small increments until the optimum effect under circumstances is reached. Dosage amounts and intervals can be adjusted individually to provide levels of the administered composition effective for the particular clinical indication being treated. This will provide a therapeutic regimen that is commensurate with the severity of the individual's disease state.

“Co-administer” it is meant that a composition described herein is administered at the same time, just prior to, or just after the administration of one or more additional therapies. The compositions provided herein can be administered alone or can be coadministered to the patient. Coadministration is meant to include simultaneous or sequential administration of the compositions individually or in combination (more than one composition). Thus, the preparations can also be combined, when desired, with other active substances (e.g. to reduce metabolic degradation).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a composition as described herein (including embodiments and examples).

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compositions of the disclosure. One of skill in the art will recognize that other pharmaceutical excipients are useful in the present disclosure.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

As used herein, the “stem cells” refers to cells which are capable of remaining in an undifferentiated state (e.g., pluripotent or multipotent stem cells) for extended periods of time in culture until induced to differentiate into other cell types having a particular, specialized function (e.g., fully differentiated cells). In embodiments, “stem cells” include embryonic stem cells (ESCs), induced pluripotent stem cells (iPSCs), adult stem cells, mesenchymal stem cells and hematopoietic stem cells. In embodiments, RPE cells are generated from pluripotent stem cells (e.g., ESCs or iPSCs).

As used herein, “induced pluripotent stem cells” or “iPSCs” are cells that can be generated from somatic cells by genetic manipulation of somatic cells, e.g., by retroviral transduction of somatic cells such as fibroblasts, hepatocytes, gastric epithelial cells with transcription factors such as Oct-3/4, Sox2, c-Myc, and KLF4 [Yamanaka S, Cell Stem Cell. 2007, 1(1):39-49; Aoi T, et al., Generation of Pluripotent Stem Cells from Adult Mouse Liver and Stomach Cells. Science. 2008 Feb. 14. (Epub ahead of print); I H Park, Zhao R, West J A, et al. Reprogramming of human somatic cells to pluripotency with defined factors. Nature 2008; 451:141-146; K Takahashi, Tanabe K, Ohnuki M, et al. Induction of pluripotent stem cells from adult human fibroblasts by defined factors. Cell 2007; 131:861-872]. Other embryonic-like stem cells can be generated by nuclear transfer to oocytes, fusion with embryonic stem cells or nuclear transfer into zygotes if the recipient cells are arrested in mitosis. In addition, iPSCs may be generated using non-integrating methods e.g., by using small molecules or RNA.

The term “embryonic stem cells” refers to embryonic cells that are capable of differentiating into cells of all three embryonic germ layers (i.e., endoderm, ectoderm and mesoderm), or remaining in an undifferentiated state. The phrase “embryonic stem cells” comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO 2006/040763) and embryonic germ (EG) cells which are obtained from the genital tissue of a fetus any time during gestation, preferably before 10 weeks of gestation. In embodiments, embryonic stem cells are obtained using well-known cell-culture methods. For example, human embryonic stem cells can be isolated from human blastocysts.

Human blastocysts are typically obtained from human in vivo preimplantation embryos or from in vitro fertilized (IVF) embryos. Alternatively, a single cell human embryo can be expanded to the blastocyst stage. For example, for the isolation of human ES cells the zona pellucida is removed from the blastocyst and the inner cell mass (ICM) is isolated by a procedure in which the trophectoderm cells are lysed and removed from the intact ICM by gentle pipetting. The ICM is then plated in a tissue culture flask containing the appropriate medium which enables its outgrowth. Following 9 to 15 days, the ICM derived outgrowth is dissociated into clumps either by a mechanical dissociation or by an enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 4-7 days. For further details on methods of preparation human ES cells, see Reubinoff et al. Nat Biotechnol 2000, May: 18(5): 559; Thomson et al., [U.S. Pat. No. 5,843,780; Science 282: 1145, 1998; Curr. Top. Dev. Biol. 38: 133, 1998; Proc. Natl. Acad. Sci. USA 92: 7844, 1995]; Bongso et al., [Hum Reprod 4: 706, 1989]; and Gardner et al., [Fertil. Steril. 69: 84, 1998].

It is appreciated that commercially available stem cells can also be used in aspects and embodiments of the present disclosure. Human ES cells may be purchased from the NIH human embryonic stem cells registry, www.grants.nih.govstem_cells or from other hESC registries. Non-limiting examples of commercially available embryonic stem cell lines are HAD-C 102, ESI, BGO 1, BG02, BG03, BG04, CY12, CY30, CY92, CY1O, TE03, TE32, CHB-4, CHB-5, CHB-6, CHB-8, CHB-9, CHB-10, CHB-11, CHB-12, HUES 1, HUES 2, HUES 3, HUES 4, HUES 5, HUES 6, HUES 7, HUES 8, HUES 9, HUES 10, HUES 11, HUES 12, HUES 13, HUES 14, HUES 15, HUES 16, HUES 17, HUES 18, HUES 19, HUES 20, HUES 21, HUES 22, HUES 23, HUES 24, HUES 25, HUES 26, HUES 27, HUES 28, CyT49, RUES3, WAO 1, UCSF4, NYUES 1, NYUES2, NYUES3, NYUES4, NYUESS, NYUES6, NYUES7, UCLA 1, UCLA 2, UCLA 3, WA077 (H7), WA09 (H9), WA 13 (H13), WA14 (H14), HUES 62, HUES 63, HUES 64, CT I, CT2, CT3, CT4, MA135, Eneavour-2, WIBR 1, WIBR2, WIBR3, WIBR4, WIBRS, WIBR6, HUES 45, Shef 3, Shef 6, BINhem19, BJNhem20, SAGO 1, SAOO1.

According to some embodiments, the embryonic stem cell line is HAD-C102 or ESI.

In addition, ES cells can be obtained from other species, including mouse (Mills and Bradley, 2001), golden hamster [Doetschman et al., 1988, Dev Biol. 127: 224-7], rat [lannaccone et al., 1994, Dev Biol. 163: 288-92], rabbit [Giles et al. 1993, Mol Reprod Dev. 36: 130-8; Graves & Moreadith, 1993, Mol Reprod Dev. 1993, 30 36: 424-33], several domestic animal species [Notarianni et al., 1991, J Reprod Fertil Suppl. 43: 255-60; Wheeler 1994, Reprod Fertil Dev. 6: 563-8; Mitalipova et al., 2001, Cloning. 3: 59-67] and non-human primate species (Rhesus monkey and marmoset) [Thomson et al., 1995, Proc Natl Acad Sci USA. 92: 7844-8; Thomson et al., 1996, Biol Reprod. 55: 254-9].

Extended blastocyst cells (EBCs) can be obtained from a blastocyst of at least nine days post fertilization at a stage prior to gastrulation. Prior to culturing the blastocyst, the zona pellucida is digested [for example by Tyrode's acidic solution (Sigma Aldrich, St Louis, MO, USA)] so as to expose the inner cell mass. The blastocysts are then cultured as whole embryos for at least nine days (and preferably not longer than fourteen days) post fertilization (i.e., prior to the gastrulation event) in vitro using standard embryonic stem cell culturing methods.

Another method for preparing ES cells is described in Chung et al., Cell Stem Cell, Volume 2, Issue 2, 113-117, 7 Feb. 2008. This method comprises removing a single cell from an embryo during an in vitro fertilization process. The embryo is not destroyed in this process.

EG (embryonic germ) cells can be prepared from the primordial germ cells obtained from fetuses of about 8-11 weeks of gestation (in the case of a human fetus) using laboratory techniques known to anyone skilled in the arts. The genital ridges are dissociated and cut into small portions which are thereafter disaggregated into cells by mechanical dissociation. The EG cells are then grown in tissue culture flasks with the appropriate medium. The cells are cultured with daily replacement of medium until a cell morphology consistent with EG cells is observed, typically after 7-30 days or 1-4 passages. For additional details on methods of preparing human EG cells, see Shamblott et al., [Proc. Natl. Acad. Sci. USA 95: 13726, 1998] and U.S. Pat. No. 6,090,622 incorporated herein by reference in their entirety.

Yet another method for preparing ES cells is by parthenogenesis. The embryo is also not destroyed in the process.

The ESCs, or other pluripotent stem cell, may be expanded without feeders prior to the differentiation step. For example, feeder cell free systems can be used in ES cell culturing. Such systems utilize matrices supplemented with serum replacement, cytokines and growth factors (including IL6 and soluble IL6 receptor chimera) as a replacement for the feeder cell layer. Stem cells can be grown on a solid surface such as an extracellular matrix (e.g., MATRIGEL™, laminin or vitronectin) in the presence of a culture medium—for example the Lonza L7 system, mTeSR, StemPro, XFKSR, NUTRISTEM®). Unlike feeder-based cultures which require the simultaneous growth of feeder cells and stem cells and which may result in mixed cell populations, stem cells grown on feeder-free systems are easily separated from the surface. The culture medium used for growing the stem cells contains factors that effectively inhibit differentiation and promote their growth such as MEF-conditioned medium and bFGF. The feeder-free culture medium TeSR™-E8™ is not used in the methods and protocols described and illustrated herein.

In some embodiments, following expansion, the pluripotent stem cells, e.g., ESCs, are subjected to directed differentiation on an adherent surface (without intermediate generation of spheroid or embryoid bodies). See, for example, International Patent Application Publication No. WO 2017/072763, incorporated by reference herein in its entirety for all methods, cells, reagents, compositions, and all other information disclosed therein.

Thus, according to an aspect of the present disclosure, at least 50%, 55%, 60%, 65%, 70%, 75%, 80%, 85%, 90%, 91%, 92%, 93%, 94%, 95%, 96%, 97%, 98%, 99% or 100% of the cells which are subjected to directed differentiation on the adherent surface are undifferentiated pluripotent stem cells (PSCs), e.g., ESCs, and express markers of pluripotency. For example, at least 50%, 55%, 60%, 65%, 70%, 75%, 80%, 85%, 90%, 91%, 92%, 93%, 94%, 95%, 96%, 97%, 98%, 99% or 100% of the cells are Oct4+TRA-I-60+. The non-differentiated PSCs may express other markers of pluripotency, such as NANOG, Rex-1, alkaline phosphatase, Sox2, TDGFO, SSEA-3, SSEA-4, SSEA-5, OCT4, TRA-1-60 and/or TRA-1-81.

In one example differentiation protocol, the non-differentiated pluripotent stem cells are differentiated towards the RPE cell lineage on an adherent surface using suspendable cell support matrix; e.g. microcarriers (MC), in dynamic suspension. For example, Star-Plus Microcarriers from SoloHill® may be used. A differentiating agent such as a member of the transforming growth factor β (TGFβ) superfamily, (e.g. TGF 1, TGF2, and TGF 3 subtypes, as well as homologous ligands including activin (e.g., activin A, activin B, and activin AB), nodal, anti-mullerian hormone (AMH), some bone morphogenetic proteins (BMP), e.g. BMP2, BMP3, BMP4, BMP5, BMP6, and BMP7, and growth and differentiation factors (GDF)) may be used.

According to some embodiments, a differentiating agent such as nicotinamide (NIC) may be used at concentrations of between about 1-100 mM, 5-50 mM, 5-20 mM, and e.g. 10 mM. The concentration may be any value or subrange within the recited ranges, including endpoints.

NIC, also known as “niacinamide” or NA, is the amide derivative form of Vitamin B3 (niacin) which is thought to preserve and improve beta cell function. NIC is essential for growth and the conversion of foods to energy, and it has been used in arthritis treatment and diabetes treatment and prevention. NA has the chemical formula C₆H₆N₂₀ and the following structure:

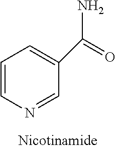

According to some embodiments, the nicotinamide is a nicotinamide derivative or a nicotinamide mimic. The term “derivative of nicotinamide (NA)” as used herein denotes a compound which is a chemically modified derivative of the natural NA. In one embodiment, the chemical modification may be a substitution of the pyridine ring of the basic NA structure (via the carbon or nitrogen member of the ring), via the nitrogen or the oxygen atoms of the amide moiety. When substituted, one or more hydrogen atoms may be replaced by a substituent and/or a substituent may be attached to a N atom to form a tetravalent positively charged nitrogen. Thus, the nicotinamide of the present invention includes a substituted or non-substituted nicotinamide. In another embodiment, the chemical modification may be a deletion or replacement of a single group, e.g. to form a thiobenzamide analog of NA, all of which being as appreciated by those versed in organic chemistry. The derivative in the context of the invention also includes the nucleoside derivative of NA (e.g. nicotinamide adenine). A variety of derivatives of NA are described, some also in connection with an inhibitory activity of the PDE4 enzyme (WO 03/068233; WO 02/060875; GB2327675A), or as VEGF-receptor tyrosine kinase inhibitors (WO 01/55114). For example, the process of preparing 4-aryl-nicotinamide derivatives (WO 05/014549). Other exemplary nicotinamide derivatives are disclosed in WO 01/55114 and EP2128244. Each of these references is incorporated herein by reference in its entirety.

Nicotinamide mimics include modified forms of nicotinamide, and chemical analogs of nicotinamide which recapitulate the effects of nicotinamide in the differentiation and maturation of RPE cells from pluripotent cells. Exemplary nicotinamide mimics include benzoic acid, 3-aminobenzoic acid, and 6-aminonicotinamide. Another class of compounds that may act as nicotinamide mimics are inhibitors of poly(ADP-ribose) polymerase (PARR). Exemplary PARP inhibitors include 3-aminobenzamide, Iniparib (BSI 201), Olaparib (AZD-2281), Rucaparib (AG014699, PF-01367338), Veliparib (ABT-888), CEP 9722, MK 4827, and BMN-673.

Additional contemplated differentiation agents include for example noggin, antagonists of Wnt (Dkk1 or IWR1e), nodal antagonists (Lefty-A), retinoic acid, taurine, GSK3b inhibitor (CHIR99021) and notch inhibitor (DAFT).

The term “retinal pigment epithelium” or “RPE,” also known as “pigmented layer of retina,” refers to the pigmented layer of cells outside the retina. The RPE layer is located between the Bruch's membrane (choroid inner border) and the photoreceptors. The RPE is an intermediate for supplying nutrients to the retina, and assists in numerous functions, including retina development, absorption of light, secretion of growth factors, and mediating the immune response of the eye. Dysfunction of the RPE may lead to vision loss or blindness in conditions including retinitis pigmentosa, diabetic retinopathy, West Nile virus, and macular degeneration.

As used herein the phrase “markers of mature RPE cells” refers to antigens (e.g., proteins) that are elevated (e.g., at least 2-fold, at least 5-fold, at least 10-fold) in mature RPE cells with respect to non RPE cells or immature RPE cells.

As used herein the phrase “markers of RPE progenitor cells” refers to antigens (e.g., proteins) that are elevated (e.g., at least 2-fold, at least 5-fold, at least 10-fold) in RPE progenitor cells when compared with non RPE cells.

According to some embodiments, the RPE cells have a morphology similar to that of native RPE cells which form the pigment epithelium cell layer of the retina. For example, the cells may be pigmented and have a characteristic polygonal shape.

According to still other embodiments, the RPE cells are capable of treating diseases such as macular degeneration.

According to additional embodiments, the RPE cells fulfill at least 1, 2, 3, 4 or all of the requirements listed herein above.

The terms “disease” or “condition” refer to a state of being or health status of a patient or subject capable of being treated with the compositions or methods provided herein. Age-related Macular Degeneration or AMD is a progressive chronic disease of the central retina and a leading cause of vision loss worldwide. Most visual loss occurs in the late stages of the disease due to one of two processes: neovascular (“wet”) AMD and geographic atrophy (GA, “dry”). In GA, progressive atrophy of the retinal pigment epithelium, choriocapillaris, and photoreceptors occurs. The dry form of AMD is more common (85-90% of all cases), but may progress to the “wet” form, which, if left untreated, leads to rapid and severe vision loss. The estimated prevalence of AMD is 1 in 2,000 people in the US and other developed countries. This prevalence is expected to increase together with the proportion of elderly in the general population. The risk factors for the disease include both environmental and genetic factors. The pathogenesis of the disease involves abnormalities in four functionally interrelated tissues, i.e., retinal pigment epithelium (RPE), Bruch's membrane, choriocapillaris and photoreceptors. However, impairment of RPE cell function is an early and crucial event in the molecular pathways leading to clinically relevant AMD changes. There is currently no approved treatment for dry-AMD. Prophylactic measures include vitamin/mineral supplements. These reduce the risk of developing wet AMD but do not affect the development of progression of geographic atrophy (GA).

A non-limiting list of diseases for which the effects of treatment may be measured in accordance with the methods provided herein comprises retinitis pigmentosa, lebers congenital amaurosis, hereditary or acquired macular degeneration, age related macular degeneration (AMD), geographic atrophy (GA), Best disease, retinal detachment, gyrate atrophy, choroideremia, pattern dystrophy as well as other dystrophies of the RPE, Stargardt disease, RPE and retinal damage due to damage caused by any one of photic, laser, inflammatory, infectious, radiation, neo vascular or traumatic injury, retinal dysplasia, retinal atrophy, retinopathy, macular dystrophy, cone dystrophy, cone-rod dystrophy, Malattia Leventinese, Doyne honeycomb dystrophy, Sorsby's dystrophy, pattern/butterfly dystrophies, Best vitelliform dystrophy, North Carolina dystrophy, central areolar choroidal dystrophy, angioid streaks, toxic maculopathy, pathologic myopia, retinitis pigmentosa, and macular degeneration. In embodiments, the disease is dry AMD. In embodiments, the disease is GA.

“Geographic atrophy” or “GA” or “atrophic retina,” also known as atrophic age-related macular degeneration (AMD) or advanced dry AMD, is an advanced form of age-related macular degeneration that can result in the progressive and irreversible loss of retina (photoreceptors, retinal pigment epithelium, choriocapillaris), which may lead to a loss of visual function over time.

In embodiments, the RPE defects may result from one or more of advanced age, cigarette smoking, unhealthy body weight, low intake of antioxidants, or cardiovascular disorders. In other embodiments, the RPE defects may result from a congenital abnormality. “Retinal pigment epithelium cells”, “RPE cells”, “RPEs”, which may be used interchangeably as the context allows, refers to cells of a cell type that is for example, functionally, epigenetically, or by expression profile similar to that of native RPE cells which form the pigment epithelium cell layer of the retina (e.g., upon transplantation, administration or delivery within an eye, they exhibit functional activities similar to those of native RPE cells).

As used herein, the term “OpRegen” refers to a lineage-restricted human RPE cell line. The RPE cells are derived under differentiation media supplemented with Activin A, a transforming growth factor beta (TGF-b) family and nicotinamide to enrich the RPE population. OPREGEN® is a single cell suspension formulated either in ophthalmic Balanced Salt Solution (BSS Plus) or as a ready to administer (RTA) thaw and inject (TAI) formulation in CryoStor® 5.

As used herein, the term “intermediate cell bank” or “ICB” refers to a stock of cells that has been frozen in aliquots at an intermediate stage in production. In embodiments, the intermediate cell bank referred to herein includes RPE cells frozen after differentiation of PSCs into RPE cells, but before expansion of the RPE cells. The ICB can be thawed and used to inoculate cultures for expansion of RPE cells, e.g., expansion on a suspendable cell support matrix. Thawing and inoculation may be days, weeks, months, or even years after freezing of the cells.

As used herein, the term “suspendable cell support matrix” refers to a suspendable support matrix that allows adherent cells to grow in dynamic or static cell culture, and can stay in suspension with gentle mixing. An example of a suspendable cell support matrix is a microcarrier.

As used herein, the term “microcarrier” or “MC” refers to a suspendable support matrix that allows adherent cells to grow in dynamic or static cell culture, and can stay in suspension with gentle mixing. Microcarriers can be composed of including, but not limited to, polystyrene, surface-modified polystyrene, chemically modified polystyrene, cross-linked dextran, cellulose, acrylamide, collagen, alginate, gelatin, glass, DEAE-dextran, or a combination thereof. Microcarriers can be coated with a biological support matrix, including, but not limited to, laminin, Matrigel, collagen, poly-lysine, poly-L-lysine, poly-D-lysine, vitronectin, fibronectin, tenascin, dextran, a peptide, or a combination thereof. Many different types of microcarriers are commercially available, including, but not limited to, HyQSphere (HyClone), Hillex (SoloHill Engineering), and Low Concentration Synthemax® II (Corning) brands. Microcarriers can be made from cross-linked dextran such as the Cytodex brand (GE Healthcare). Microcarriers can be spherical and smooth, can have microporous surfaces, such as CYTOPORE brand (GE Healthcare), and/or can be rod-shaped carriers such as DE-53 (Whatman). Microcarriers can be impregnated with magnetic particles that may help in cell separation from beads (e.g., GEM particles from Global Cell Solutions). Chip-based microcarriers such as the pHex product (Nunc) provide a flat surface for cell growth while maintaining the high surface to volume ratio of traditional microcarriers. The properties of microcarriers may significantly affect expansion rates and cell multi- or pluripotency.

In embodiments, microcarriers (MC) concentration can be about 10 cm²/mL. In embodiments, the MC concentration is 10 cm²/mL. In some embodiments, the MC concentration is from about 1 cm²/mL to about 30 cm²/mL, or from about 1 cm²/mL to about 20 cm²/mL, or from about 1 cm²/mL to about 10 cm²/mL, or from about 1 cm²/mL to about 5 cm²/mL. In some embodiments, the MC concentration is about 1 cm²/mL, or about 2 cm²/mL, or about 3 cm²/mL, or about 4 cm²/mL, or about 5 cm²/mL, or about 6 cm²/mL, or about 7 cm²/mL, or about 8 cm²/mL, or about 9 cm²/mL, or about 10 cm²/mL, or about 11 cm²/mL, or about 12 cm²/mL, or about 13 cm²/mL, or about 14 cm²/mL, or about 15 cm²/mL, or about 16 cm²/mL, or about 17 cm²/mL, or about 18 cm²/mL, or about 19 cm²/mL, or about 20 cm²/mL, or more. In embodiments, the MC concentration is at a range of 5-10 cm²/mL.

In embodiments, the suspendable cell support matrix is not coated. In embodiments, the suspendable cell support matrix is not treated. In embodiments, the suspendable cell support matrix is treated or coated to promote cell adhesion. Surface chemistry modifications can improve cell adhesion including, but not limited to, methods of applying positive or negative charges, or coating with extracellular matrix proteins such as laminin or vitronectin.

As used herein, the term “bioreactor” refers to any system that can support a biologically active environment. A bioreactor may be an open or closed system, anaerobic or aerobic. Bioreactors may be continuous or stationary flow. The bioreactor can be fed continuously or by batch. The bioreactor may monitor for levels, e.g., of dissolved oxygen (DO), pH, and for flow of gases including N₂, O₂, CO₂, and air. The bioreactor may include a means to mix or agitate the cell suspension, for example by any type of impeller or agitator within the bioreactor, or by a rocking platform to provide dynamic culture conditions. The bioreactor may be single use.

As used herein, the term “population doubling level” is the total number of times the cells in a given population have doubled during in vitro culture. The mathematical expression for the population doubling is: log 2(live cells harvested/live cells seeded). For example, if 1 million live cells were seeded and 8 million live cells were harvested, log 2(8/1)=3. Meaning, the averaged cell population doubling was 3.

### Methods of RPE Cell Expansion

Embodiments herein generally relate to methods for the expansion of retinal pigment epithelial (RPE) cells, including the use of a suspendable cell support matrix, such as a microcarrier.

In some embodiments, the RPE cells are differentiated from human embryonic stem cells (hESC). In some embodiments, the RPE cells are differentiated from human pluripotent stem cells (iPSC).

According to certain embodiments, the differentiation is effected as follows: (a) expansion of feeder-free hESCs (FF hESCs) in a highly controlled culturing system; (b) FF monolayer directed differentiation of cells obtained from step a) in a medium comprising a member of the TGFβ superfamily (e.g. activin A) and a differentiating agent (e.g. nicotinamide); (c) RPE expansion on gelatin coated vessels; and (d) RPE at second passage cultured on a suspendable cell support matrix. Step (a) may be effected in the absence of the member of the TGFβ superfamily (e.g. activin A). Non-limiting examples of the production of RPE cells from pluripotent stem cells may be found in International Patent Application Nos. WO 2021/242788 A1, WO 2017/021973 A1, WO 2017/021972 A1, WO 2017/017686 A1, WO 2016/108239 A9, WO 2016/108239 A9, WO 2016/108239 A9, WO 2008/129554 A1, WO 2006/070370 A3, WO 2019/028088 A1, WO 2021/242788 A1, WO/2020/223226, and WO 2013/114360 A1. Each of these references is incorporated by reference herein in its entirety, for all compositions, reagents and cells, as well as all methods, methods of manufacturing and methods of using compositions, reagents, and cells, and all other information disclosed therein.

In some embodiments, the medium in step (a) is completely devoid of a member of the TGFβ superfamily. In other embodiments, the level of TGFβ superfamily member in the medium is less than 20 ng/mL, less than 10 ng/mL, less than 1 ng/mL or even less than 0.1 ng/mL. The concentration may be any value or subrange within the recited ranges, including endpoints.

The above described protocol may be followed by a validation procedure. The validation procedure may include the following criteria: (a) a high purity of RPE cells having over 95% purity as measured by CRALBP/PMEL17 flow cytometry; (b) generation of a polarized monolayer post thawing having net transepithelial electrical resistance (TEER) >100Ω/cm² and polarized secretion of PEDF and VEGF; and/or (c) no residual stem cells, e.g. hESCs, confirmed by high accuracy Fuzzy C-Mean (FCM) method, lacking TRA-1-60/Oct-4 as measured by flow cytometry.

Cell suspension obtained using the above described protocol can then be transplanted, e.g. into a patient in need thereof. The cells can repopulate a vast area, locating at needed harmed spaces to expand by committed RPE cell expansion until reaching contact inhibition. The cells can generate a mature and polarized RPE layer with barrier function for batch release (BR) potency as measured by TEER and polarized PEDF and VEGF secretion. The cell suspension obtained is ready to be thawed and injected into patients, with no need for cell preparation prior to surgery.

The above described protocol may yield approximately 2500 vials having 5×10⁹ RPE cells per 3 L bioreactor. The number of bioreactors and volume of bioreactors can be increased with relative ease.

According to some embodiments, at least 50%, 60% 70%, 80%, 85%, 87%, 89%, 90%, or 95% of the cells express cellular retinaldehyde binding protein (CRALBP), as measured by immunostaining. For example, between 95-100% of the cells express CRALBP. The percentage may be any value or subrange within the recited ranges, including endpoints.

According to another embodiment, at least 50%, 60% 70%, 80%, 85%, 87%, 89%, 90%, or 95% of the cells express cellular Melanocytes Lineage-Specific Antigen GP100 (PMEL17), as measured by immunostaining. For example, between 95-100% of the cells express PMEL17. The percentage may be any value or subrange within the recited ranges, including endpoints.

In an aspect, provided herein is a method for the expansion of retinal pigment epithelial (RPE) cells, the method including: providing a population of RPE cells; inoculating a medium containing a first suspendable cell support matrix with the population of RPE cells; and expanding the population of RPE cells on the first suspendable cell support matrix in dynamic suspension to provide an expanded population of RPE cells. In embodiments, the population of RPE cells was differentiated from pluripotent stem cells prior to the providing step.

In an embodiment, the population of RPE cells is expanded on a solid surface under static conditions. In some embodiments, the solid surface is a culture plate. In some embodiments, the solid surface is a culture flask. In some embodiments, the solid surface is a multi-well culture dish.

In an embodiment, the population of RPE cells was expanded on a solid surface under dynamic conditions prior to the first inoculation step. In some embodiments, the solid surface contains a second suspendable cell support matrix.

In some embodiments, the solid surface is coated. In some embodiments, the solid surface is coated by laminin, Matrigel, collagen, poly-lysine, poly-L-lysine, poly-D-lysine, vitronectin, fibronectin, tenascin, dextran, a peptide, or a combination thereof. In some embodiments, the solid surface is coated with laminin. In some embodiments, the solid surface is coated with Matrigel. In some embodiments, the solid surface is coated with collagen. In some embodiments, the solid surface is coated with poly-lysine. In some embodiments, the solid surface is coated with poly-L-lysine. In some embodiments, the solid surface is coated with poly-D-lysine. In some embodiments, the solid surface is coated with vitronectin. In some embodiments, the solid surface is coated with fibronectin. In some embodiments, the solid surface is coated with tenascin. In some embodiments, the solid surface is coated with dextran. In some embodiments, the solid surface is coated with a peptide.

In embodiments, the population of RPE cells is provided from an intermediate cell bank. In some embodiments, the population of RPE cells was expanded on the solid surface under static conditions for one passage prior to providing the RPE cells.

In embodiments, the first suspendable cell support matrix is uncoated. In embodiments, the suspendable cell support matrix is coated.

In embodiments, the differentiation of the population of RPE cells from pluripotent stem cells includes: i) expansion of pluripotent stem cells on a solid surface under conditions that maintain pluripotency of the pluripotent stem cells to provide expanded pluripotent stem cells; and ii) differentiating the expanded pluripotent stem cells in a medium comprising a differentiating agent and optionally a growth factor for a period of time to provide the population of RPE cells.

In some embodiments, the solid surface is a culture plate. In some embodiments, the solid surface includes a second suspendable cell support matrix. In some embodiments, the pluripotent stem cells are expanded in dynamic culture.

In embodiments, method step ii) includes differentiating the expanded pluripotent stem cells. In some embodiments, the pluripotent stem cells are expanded on a third suspendable cell support matrix in dynamic culture. In some embodiments, the expanded pluripotent stem cells from step i) remain attached to the second suspendable cell support matrix in step ii). In some embodiments, method step ii) includes differentiating the expanded pluripotent stem cells on a culture plate in static culture.

In embodiments, the pluripotent stem cells are grown into a monolayer adherent to the second suspendable cell support matrix and/or third suspendable cell support matrix. In some embodiments, the pluripotent stem cells are grown into a monolayer adherent to the second suspendable cell support matrix. In some embodiments, the pluripotent stem cells are grown into a monolayer adherent to the third suspendable cell support matrix. In some embodiments, the pluripotent stem cells are grown into a monolayer adherent to the second and third suspendable cell support matrices.

In embodiments, the conditions for maintaining pluripotency of the pluripotent stem cells are feeder cell free. In some embodiments, the conditions for maintaining pluripotency comprise a feeder cell population.

In embodiments, the differentiating reagent is nicotinamide.

In embodiments, the growth factor is a member of the TGFβ family. In some embodiments, the member of the transforming growth factor-B (TGFβ) superfamily is TGF 1, TGF2, and TGF 3 subtypes, as well as homologous ligands including activin (e.g., activin A, activin B, and activin AB), nodal, anti-mullerian hormone (AMH), some bone morphogenetic proteins (BMP), e.g. BMP2, BMP3, BMP4, BMP5, BMP6, and BMP7, and growth and differentiation factors (GDF)). According to a specific embodiment, the member of the transforming growth factor-B (TGFβ) superfamily is activin A.

In embodiments, the first suspendable cell support matrix, second suspendable cell support matrix, and/or third suspendable cell support matrix is composed of polystyrene, surface-modified polystyrene, chemically modified polystyrene, cross-linked dextran, cellulose, acrylamide, collagen, alginate, gelatin, glass, DEAE-dextran, or a combination thereof. In some embodiments, the suspendable cell support matrix is composed of polystyrene. In some embodiments, the suspendable cell support matrix is composed of surface-modified polystyrene. In some embodiments, the suspendable cell support matrix is composed of chemically modified polystyrene. In some embodiments, the suspendable cell support matrix is composed of cross-linked dextran. In some embodiments, the suspendable cell support matrix is composed of cellulose. In some embodiments, the suspendable cell support matrix is composed of acrylamide. In some embodiments, the suspendable cell support matrix is composed of collagen. In some embodiments, the suspendable cell support matrix is composed of alginate. In some embodiments, the suspendable cell support matrix is composed of gelatin. In some embodiments, the suspendable cell support matrix is composed of glass. In some embodiments, the suspendable cell support matrix is composed of DEAE-dextran.

In embodiments, the first suspendable cell support matrix, second suspendable cell support matrix, and/or third suspendable cell support matrix is uncoated. In some embodiments, the first suspendable cell support matrix, second suspendable cell support matrix, and/or third suspendable cell support matrix is coated. In some embodiments, the suspendable cell support matrix is coated by laminin, Matrigel, collagen, poly-lysine, poly-L-lysine, poly-D-lysine, vitronectin, fibronectin, tenascin, dextran, a peptide, or a combination thereof. In some embodiments, suspendable cell support matrix is coated with laminin. In some embodiments, the suspendable cell support matrix is coated with Matrigel. In some embodiments, the suspendable cell support matrix is coated with collagen. In some embodiments, the suspendable cell support matrix is coated with poly-lysine. In some embodiments, the suspendable cell support matrix is coated with poly-L-lysine. In some embodiments, the suspendable cell support matrix is coated with poly-D-lysine. In some embodiments, the suspendable cell support matrix is coated with vitronectin. In some embodiments, the suspendable cell support matrix is coated with fibronectin. In some embodiments, the suspendable cell support matrix is coated with tenascin. In some embodiments, the suspendable cell support matrix is coated with dextran. In some embodiments, the suspendable cell support matrix is coated with a peptide.

In embodiments, the first suspendable cell support matrix, second suspendable cell support matrix, and/or third suspendable cell support matrix is spherical, ellipsoidal, rod-shaped, disc-shaped, porous, non-porous, smooth, flat, or a combination thereof. In some embodiments, the suspendable cell support matrix is spherical. In some embodiments, the suspendable cell support matrix is ellipsoidal. In some embodiments, the suspendable cell support matrix is rod-shaped. In some embodiments, the suspendable cell support matrix is disc-shaped. In some embodiments, the suspendable cell support matrix is porous. In some embodiments, the suspendable cell support matrix is non-porous. In some embodiments, the suspendable cell support matrix is smooth. In some embodiments, the suspendable cell support matrix is flat.

In embodiments, the first suspendable cell support matrix, second suspendable cell support matrix, and third suspendable cell support matrices are the same. In some embodiments, at least two of the first suspendable cell support matrix, second suspendable cell support matrix, and third suspendable cell support matrix are the same. In some embodiments, the first suspendable cell support matrix, second suspendable cell support matrix, and third suspendable cell support matrix are different.

In embodiments, the suspendable cell support matrix is a microcarrier. In some embodiments the first suspendable cell support matrix is a first microcarrier. In some embodiments the second suspendable cell support matrix is a second microcarrier. In some embodiments, the third suspendable cell support matrix is a third microcarrier.

In embodiments, the population of RPE cells has a population doubling level between 2-4 during P0 (initial growth phase after differentiation, or after inoculation from ICB), 2-3 during P1 (a first passage after P0), and 1-2 during P2 (second passage).

In some embodiments, expansion of RPE cells as described herein may result in a greater number of cells grown over a given amount of time. For example, during the course of one year, without ICB 4 batches may be grown per suit if 2 different batches are grown at the same location; in contrast, with ICB in one year 10 batches per suit of the same size may be grown, e.g., 3 times the amount without growing 2 batches at the same time. In some embodiments, the number of RPE cells at the end of expansion is about 2 times higher with the use of an ICB compared to without. In some embodiments, the number of RPE cells at the end of expansion is about 3 times higher with the use of an ICB. In some embodiments, the number of RPE cells at the end of expansion is about 4 times higher with the use of an ICB. In some embodiments, the cost of an RPE expansion growth is less with the use of an ICB than without the use of an ICB.

In embodiments, the conditions for expansion of the RPE cells include maintaining % dissolved oxygen above 30%.

In embodiments, the conditions for expansion of the RPE cells include initial a growth media volume starting at about 50% of total system growth chamber volume. In embodiments, a growth media volume of about 10% to about 25%, e.g., about 16.6%, of total system growth chamber volume is added periodically. In embodiments, the growth media volume is added every 2 to 4 days.

In embodiments, the RPE cells are characteristic of mature RPE cells. In some embodiments, the mature RPE cells are double positive for cellular retinaldehyde-binding protein (CRALBP) and premelanosome protein (PMEL17) at greater than 95% as measured by flow cytometry. In some embodiments, the mature RPE cells generate a polarized monolayer post thawing having net transepithelial electrical resistance (TEER) >100Ω/cm² and polarized secretion of PEDF and VEGF.

In some embodiments, the mature RPE cells are cryopreserved and ready for administration to a subject upon thawing. In embodiments, the RPE cells are cryopreserved in a cryopreservation medium. In embodiments, the cryopreservation medium includes a cryoprotective agent, for example glycerol, sucrose, dimethyl sulfoxide (DMSO), or other suitable cryoprotective agent. In embodiments, the cryoprotective agent includes glycerol. In embodiments, the cryoprotective agent includes sucrose. In embodiments, the cryoprotective agent includes DMSO. In embodiments, the cryoprotective agent includes dextran.

In embodiments, the cryopreservation medium includes about 0.1% to about 40% of the cryoprotective agent. In embodiments, the cryopreservation medium includes about 0.10% to about 30% of the cryoprotective agent. In embodiments, the cryopreservation medium includes about 0.1% to about 20% of the cryoprotective agent. In embodiments, the cryopreservation medium includes about 0.10% to about 10% of the cryoprotective agent. In embodiments, the cryopreservation medium includes about 0.1% to about 5% of the cryoprotective agent. In embodiments, the cryopreservation medium includes about 1% to about 40% of the cryoprotective agent. In embodiments, the cryopreservation medium includes about 1% to about 30% of the cryoprotective agent. In embodiments, the cryopreservation medium includes about 1% to about 20% of the cryoprotective agent. In embodiments, the cryopreservation medium includes about 1% to about 10% of the cryoprotective agent. In embodiments, the cryopreservation medium includes about 1% to about 5% of the cryoprotective agent. The percentage may be measured as weight of cryoprotective agent per volume of medium. The percentage may be measured as volume of cryoprotective agent per volume of medium. The percentage may be any value or subrange within the recited ranges, including endpoints.

In some embodiments, the mature RPE cells contain <0.01% pluripotent stem cells as confirmed by a high accuracy flow cytometry method (FCM), and are negative for TRA-1-60/Oct-4 as measured by flow cytometry.

In some embodiments, the RPE cells generate a polygonal monolayer at every passage during their expansion post-differentiation until formulation, and after injection as a cell suspension.

In embodiments, one or more steps of the method is performed in a single-use bioreactor. In embodiments, the expansion of RPE cells is performed in a single-use bioreactor. In embodiments, the differentiation of RPE cells is performed in a single-use bioreactor.

### Methods of Treatment

Embodiments herein generally relate to methods, compositions of matter, and devices for treating diseases and illnesses of the eye, including retinal conditions such as macular degeneration.

In an aspect, a method of treating a disorder or disease of the eye, the method including transplanting into the retinal tissue of a patient in need thereof a pharmaceutical composition containing RPE cells generated from a method of expansion of retinal pigment epithelial (RPE) cells as described herein

In embodiments, the disorder or disease of the eye is age-related macular degeneration (AMD), hereditary macular degenerations including Best disease (the early onset form of vitelliform macular dystrophy), or a subtype of retinitis pigmentosa (RP). In embodiments, the disorder or disease of the eye is age-related macular degeneration (AMD). In embodiments, the disorder or disease of the eye is hereditary macular degenerations. In embodiments, the disorder or disease of the eye is Best disease (the early onset form of vitelliform macular dystrophy). In embodiments, the disorder or disease of the eye is a subtype of retinitis pigmentosa (RP).

In embodiments, the population of RPE cells is ready for use in a patient based on a product release determination including: determining the mature RPE cells are double-positive for cellular retinaldehyde-binding protein (CRALBP) and premelanosome protein (PMEL17) at greater than 95%, as measured by flow cytometry; determining the mature RPE cells generate a polarized monolayer post thawing having net transepithelial electrical resistance (TEER) >100Ω/cm2 and polarized secretion of PEDF and VEGF; and/or the mature RPE cells comprise <0.01% pluripotent stem cells as confirmed by a high accuracy flow cytometry method (FCM), and are negative for TRA-1-60/Oct-4 as measured by flow cytometry.

In some embodiments, the RPE cells generate a polygonal monolayer at every passage during their expansion post-differentiation until formulation, and after injection as a cell suspension.

In embodiments, the concentration of mature RPE cells is in the range of 10,000-500,000 cells per 50-200 microliters. In embodiments, the concentration of mature RPE cells is in the range of 15,000-300,000 cells per 50-200 microliters. In embodiments, the concentration of mature RPE cells is in the range of 25,000-250,000 cells per 50-200 microliters. In embodiments, the concentration of mature RPE cells is in the range of 50,000-250,000 cells per 50-200 microliters. In embodiments, the concentration of mature RPE cells is in the range of 10,000-500,000 cells per 50 microliters. In embodiments, the concentration of mature RPE cells is in the range of 15,000-300,000 cells per 50 microliters. In embodiments, the concentration of mature RPE cells is in the range of 25,000-250,000 cells per 50 microliters. In embodiments, the concentration of mature RPE cells is in the range of 50,000-250,000 cells per 50 microliters. In embodiments, the concentration of mature RPE cells is in the range of 10,000-500,000 cells per 100 microliters. In embodiments, the concentration of mature RPE cells is in the range of 15,000-300,000 cells per 100 microliters. In embodiments, the concentration of mature RPE cells is in the range of 25,000-250,000 cells per 100 microliters. In embodiments, the concentration of mature RPE cells is in the range of 50,000-250,000 cells per 100 microliters. In embodiments, the concentration of mature RPE cells is in the range of 10,000-500,000 cells per 150 microliters. In embodiments, the concentration of mature RPE cells is in the range of 15,000-300,000 cells per 150 microliters. In embodiments, the concentration of mature RPE cells is in the range of 25,000-250,000 cells per 150 microliters. In embodiments, the concentration of mature RPE cells is in the range of 50,000-250,000 cells per 150 microliters. In embodiments, the concentration of mature RPE cells is in the range of 10,000-500,000 cells per 200 microliters. In embodiments, the concentration of mature RPE cells is in the range of 15,000-300,000 cells per 200 microliters. In embodiments, the concentration of mature RPE cells is in the range of 25,000-250,000 cells per 200 microliters. In embodiments, the concentration of mature RPE cells is in the range of 50,000-250,000 cells per 200 microliters. The concentration may be any value or subrange within the recited ranges, including endpoints.

In some embodiments, the concentration of mature RPE cells is about 25,000 cells per 50 microliters. In some embodiments, the concentration of mature RPE cells is about 50,000 cells per 50 microliters. In some embodiments, the concentration of mature RPE cells is about 75,000 cells per 50 microliters. In some embodiments, the concentration of mature RPE cells is about 100,000 cells per 50 microliters. In some embodiments, the concentration of mature RPE cells is about 125,000 cells per 50 microliters. In some embodiments, the concentration of mature RPE cells is about 150,000 cells per 50 microliters. In some embodiments, the concentration of mature RPE cells is about 175,000 cells per 50 microliters. In some embodiments, the concentration of mature RPE cells is about 200,000 cells per 50 microliters. In some embodiments, the concentration of mature RPE cells is about 225,000 cells per 50 microliters. In some embodiments, the concentration of mature RPE cells is about 250,000 cells per 50 microliters. In some embodiments, the concentration of mature RPE cells is about 25,000 cells per 100 microliters. In some embodiments, the concentration of mature RPE cells is about 50,000 cells per 100 microliters. In some embodiments, the concentration of mature RPE cells is about 75,000 cells per 100 microliters. In some embodiments, the concentration of mature RPE cells is about 100,000 cells per 100 microliters. In some embodiments, the concentration of mature RPE cells is about 125,000 cells per 100 microliters. In some embodiments, the concentration of mature RPE cells is about 150,000 cells per 100 microliters. In some embodiments, the concentration of mature RPE cells is about 175,000 cells per 100 microliters. In some embodiments, the concentration of mature RPE cells is about 200,000 cells per 100 microliters. In some embodiments, the concentration of mature RPE cells is about 225,000 cells per 100 microliters. In some embodiments, the concentration of mature RPE cells is about 250,000 cells per 100 microliters. In some embodiments, the concentration of mature RPE cells is about 25,000 cells per 150 microliters. In some embodiments, the concentration of mature RPE cells is about 50,000 cells per 150 microliters. In some embodiments, the concentration of mature RPE cells is about 75,000 cells per 150 microliters. In some embodiments, the concentration of mature RPE cells is about 100,000 cells per 150 microliters. In some embodiments, the concentration of mature RPE cells is about 125,000 cells per 150 microliters. In some embodiments, the concentration of mature RPE cells is about 150,000 cells per 100 microliters. In some embodiments, the concentration of mature RPE cells is about 175,000 cells per 150 microliters. In some embodiments, the concentration of mature RPE cells is about 200,000 cells per 150 microliters. In some embodiments, the concentration of mature RPE cells is about 225,000 cells per 150 microliters. In some embodiments, the concentration of mature RPE cells is about 250,000 cells per 150 microliters. In some embodiments, the concentration of mature RPE cells is 25,000 cells per 200 microliters. In some embodiments, the concentration of mature RPE cells is about 50,000 cells per 200 microliters. In some embodiments, the concentration of mature RPE cells is about 75,000 cells per 200 microliters. In some embodiments, the concentration of mature RPE cells is about 100,000 cells per 200 microliters. In some embodiments, the concentration of mature RPE cells is about 125,000 cells per 200 microliters. In some embodiments, the concentration of mature RPE cells is about 150,000 cells per 200 microliters. In some embodiments, the concentration of mature RPE cells is about 175,000 cells per 200 microliters. In some embodiments, the concentration of mature RPE cells is about 200,000 cells per 200 microliters. In some embodiments, the concentration of mature RPE cells is about 225,000 cells per 200 microliters. In some embodiments, the concentration of mature RPE cells is about 250,000 cells per 200 microliters.

In some embodiments, the pharmaceutical composition is formulated to be thawed and injected into a subject without cell preparation prior to injection.

In accordance with one embodiment, transplantation is performed via pars plane vitrectomy surgery followed by delivery of the cells through a small retinal opening into the sub-retinal space or by direct injection.

In certain embodiments, administration may comprise a vitrectomy followed by delivery of the RTA therapeutic cell composition into the subretinal space in the macular area via a cannula through a small retinotomy. A total volume of 50-100 μL cell suspension, depending on the cell dose, can be implanted in areas at potential risk for GA expansion.

In some embodiments, a single surgical procedure is performed in which the RTA therapeutic cell composition is delivered through a small retinotomy, following vitrectomy, into a subretinal space created in the macular area, along the border between areas of GA, if present, and the better preserved extra-foveal retina and RPE layer. After the placement of a lid speculum, a standard 3-port vitrectomy can be performed. This may include the placement of a 23G or 25G infusion cannula and two 23G or 25/23G ports (trocars). A core vitrectomy can then be performed with 23G or 25G instruments, followed by detachment of the posterior vitreous face. The RTA therapeutic cell composition may be injected into the subretinal space at a predetermined site within the posterior pole, preferably penetrating the retina in an area that is still relatively preserved close to the border of GA, if present.

### Pharmaceutical Compositions

In some aspects the present disclosure is drawn to cell therapeutic agents comprising retinal pigment epithelial (RPE) cells derived from pluripotent cells. Such cell therapeutic agents include, but are not intended to be limited to, OPREGEN®.

In an aspect, provided herein is a pharmaceutical composition containing RPE cells generated from a method of expansion of RPE cells as described herein.

In embodiments, the composition is frozen prior to use in a patient. In embodiments, the composition is formulated to be thawed and injected into a subject without cell preparation prior to injection.

### EXAMPLES

### Example 1

The purpose of this example is to demonstrate the membrane formation of RPE cells cultured using expansion of feeder-free hESCs (FF hESCs) in a highly controlled culturing system. The feeder-free monolayer directed differentiation of cells in a medium comprising a member of the TGFβ superfamily (e.g. activin A) and a differentiating agent (e.g. nicotinamide) can then be used for RPE expansion on gelatin coated vessels; and RPE at second passage cultured on microcarriers (MC) can be used for transplantation. The expansion of FF hESCs may be effected in the absence of the member of the TGFβ superfamily (e.g. activin A).

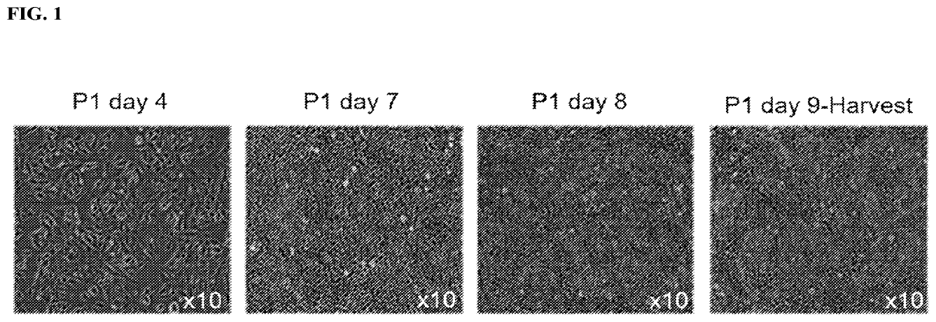

RPE cells cultured using the methods described herein can generate a polygonal monolayer membrane, even when injected as a cell suspension. The RPE cells generate such a polygonal monolayer membrane at every passage during their expansion post-differentiation and until formulation. FIG. 1 shows the RPE cells during P1 passage at RPE expansion, starting at day 4 post-seeding, becoming organized and reaching high dense polygonal morphology on day 9 (harvesting day).

### Example 2

The purpose of this example is to summarize the development of a small scale process for the expansion of RPE cells in Corning single use 0.1 L spinner flasks.

This development in small scale vessels is a preliminary step before scaling up the expansion of RPE cells in a bioreactor, for the manufacturing of OPREGEN® in controlled growth conditions. In this method, differentiated RPE were seeded on plastic MC and expanded for 1-2 passages. Several parameters were tested and optimized such as the type of plastic, the concentration of MC, and seeding cell density per area of MC.

RPE cells were expanded on MC in Spinner flasks. Several conditions were tested versus control T-flasks, in a step wise manner. Other process modifications were implemented during each run and documented along the process development. The parameter tested included:
MC type screening—Pall Sollohil (Star Plus, Plastic Plus, Hillex II) Corning Synthmax (Hi, Low, CellBind);
Seeding densities—Cells were seeded in various cells densities, the range of 60,000 to 120,000 cells/cm²;
Seed Agitation—40 RPM with 5 min rest every 30 min, 10 RPM in horizontal tube, constant 40 RPM;
Feeding regimen—Cells were fed either by media exchange or by fed batch regimen. Growth media was replaced (half volume) or added (28-56%) 2-3 times a week;
Serum concentration reduction—A Follow up to studies done in TC-flasks (EXP29 A-C), standard seeding media of 20% HS/DMEM was replaced by 2% HS/Nut minus/HSA, or 0% HS/Nut minus/HAS;
Nicotinamide—With or without addition of nicotinamide to the growth media; and
MC concentration—Several MC concentrations were tested in spinner flasks in the range of 5-20 cm²/mL (360 cm²/gr, 0.5-2 gr/spinner flask);

TABLE 1
Study design

| Study #/ | Parameter | | | |
|---|---|---|---|---|
| cells origin | tested | Description | Readouts | Comments/Conclusions |
| EXP27A/ | MC | Screening attachment to | P4 Day 7 Yield | Overall attachment and growth |
| Thawed | | microcarriers (6 types, 4 | % attachment | is better when serum > 0.5% |
| Mock IV | | serum concentrations) | Morphology | |
| P3 | | In 6-well plates | | |
| EXP27B/ | MC | Refined Screening of | P5 Day 14 Yield | Star Plus MC are better than |
| Thawed | | attachment to | Morphology | the rest but 10 times less than |
| Mock IV | | microcarriers (6 types), | | T75 control |
| P3 | | in 0.1 L spinner flasks, | | Agitation (30 to 38RPM) RPM |
| | | seeding in 20% | | and seeding volume (15 to 30 |
| | | HS/DMEM | | mL) were adjusted during run. |
| | | | | Most cells in aggregates due to |
| | | | | sub-optimal seeding |
| EXP27C/ | MC | Refined Screening of | P4 Day 14 Yield | Best yield was with Star Plus |
| Thawed | seeding | attachment to | Morphology, vitality on | MC, as good as control T75 |
| Mock 12 | densities | microcarriers (3 types, 3 | fresh and thawed cells | flask (~8) at seeding density of |
| P2 | Feeding | cell seed density of best | | 86,000/cm² |
| | | candidate), growth for 2 | | In second passage Star Plus |
| | | passages. | | and MC at 120,000/cm² had |
| | | | | better yield (6.89) than Control |
| | | | | (6.15) |
| EXP27D/ | Seed | Changing seeding Star | P1 Day 14 Yield | Yield (6.3) was better than |
| FF Mock | Agitation | Plus MC with | Morphology | Control (5.6) |
| 14 Grp H | seeding | 85,000/cm2 from 40 | | |
| P0 | densities | RPM + stops 5 min/Hr to | | |
| | Feeding | constant 40 RPM. | | |
| EXP27E/ | Seed | Testing seeding at | P2 Day 14 Yield | Thawed P2 cells passed release |
| FF Mock | Agitation | horizontal tube at 10 | Morphology | criteria for recovery potency |
| 14 Grp H | | RPM for 3 Hrs. | Recovery, purity potency | and purity |
| P1 | | | | |
| EXP27F/ | Feeding | Testing Fed Batch Vs. ½ | P2 Day 14 Yield | Thawed P2 cells passed release |
| EXP27D | | media exchange. Second | Morphology | criteria for recovery potency |
| FF Mock | | passage on Spinner | Recovery, | and purity. |
| 14 Grp H | | Flasks, seeding at | purity potency | Fed Batch was chosen |
| P1 | | 100,000/cm2 | | |
| EXP27G | [Serum] | Testing seeding media | P4 Day 14 Yield | Thawed RPE cannot grow on |
| Thawed | | without serum Nut(−) | Morphology | MC without serum (EXP29A-D |
| FF Mock | | HSA + Nic Vs. | | studies investigated the seeding |
| 14 Grp O | | 20% HS/DMEM | | and growth of RPE without |
| P2. P3 on | | | | serum in TC flasks) |
| T25 | | | | |
| (EXP29B) | | | | |
| EXP27H | +/− Nic | Thawing ICB and | Day 13 Yield | P2 cells grow well with or |
| Thawed | MC | expanding on MC with | Morphology | without Nic |
| FF Mock | concentration | or without Nic for 3 | | 5-10 cm2/ml of beads is most |
| 14 Grp H | | passages | | efficient growth conditions |
| ICB PO | | In 3 MC concentrations | | in 0.1 L and 0.5 L spinner flasks |
| | | In 0.1 L and 0.5 L spinner | | |
| | | flasks | | |
| EXP27J | Feeding | Thawing ICB and | Day 13 Yield | P2 cells grow well with or |
| Thawed | +/− Nic | expanding on MC with | Morphology | without Nic |
| FF Mock | [Serum] | or without Nic for 2 | | Can be seeded with Nut (-) |
| 14 Grp H | seeding | passages Seeding with | | HSA+ 2% HS |
| ICB P0 | densities | 20% HS or 2% HS | | |
| | | seeding at 100,000/cm2 | | |
| EXP29D | [Serum] | Thawing ICB and | P1 day 16 | Cells can be thawed and seeded |
| Thawed | seeding | expanding on MC with | P2 day 11 Yield | with 2% HS and grown for 2 |
| FF Mock | densities | 20% HS/DNEN or 2% | | passages |
| 14 Grp H | | HS in Nut(−)HSA for 2 | | |
| ICB P0 | | passages | | |
| EXP27K | [Serum] | Thawing ICB without | P1 day 11 yield, pH | Cells can be thawed without |
| Thawed | | washing the CS5% and | | washing and seeded with 2% HS |
| FF Mock | | expanding on MC with | | and grown for 2 passages with |
| 14 Grp H | | 20% HS/DNEN or 2% | | and without nic, pH along P1- |
| ICB P0 | | HS in Nut(−)HSA for 2 | | P2 was 7.1-7.3 without Nic and |
| | | passages | | 7.3-7.7 with Nic and in all |
| | | | | conditions in spinner flasks |
| EXP27L | +/− Nic | Growing FF RPE | P0 day 15 | Cells can be grown for the entire |
| FF live run | MC | differentiation. | P1, P2 day 10 | expansion phase with and |
| | concentration | P0 P1-P2 expansion on | Yield Recovery | without nic on MC. Optimal |
| | | MC +/− Nic, in P1-P2, in | Purity | MC concentration in the range |
| | | 1x and 2x MC | | of 5-10 cm2/mL |
| | | concentrations | | |

### Experimental Procedure

RPE cells were thawed directly or expanded in T-flasks before seeding on MC of different types (Table 2) in 6-Well plate wells (EXP27A) or in 0.1 L Corning spinner flasks (EXP27 B-C, G-K).

Unfrozen sources of differentiated RPE cells that were derived from feeder free hESCs, were expanded on Star plus MC (Table 2—line 1) in spinner flasks (EXP27D-F, L).

Cells were counted using the NC-200 cell counter and yield was calculated by dividing with the number of seeded cells.

Cells morphology and the levels of lactate and glucose were examined along passage.

At end of passage RPE cultures of each condition were harvested and yield was compared to control flask. Yield and morphology were compared with control T-flask in one or more consecutive passages.

In some experiments, frozen cell samples at end of passage were tested for recovery, identity and potency

Screening for suitable MC type

TABLE 2
Micro carriers tested in the EXP27 studies.

| | | | | | Diameter |
|---|---|---|---|---|---|
| | MC Type and Cat# | Company | Charge/Coating | Matrix | (μM) |
| 1 | Solohill Star-Plus SP102- | PALL | Positive (electric) | Crosslinked | 125-212 |
| | 1521 | | | Polystyrene | |
| 2 | Solohill Hillex ®II H112- | PALL | Positive (chemical) | Modified polystyrene | 160-200 |
| | 170 | | | | |
| 3 | Solohill Plastic Plus PP102- | PALL | Positive | Cross-linked | 125-212 |
| | 1521 | | | polystyrene, cationic | |
| | | | | charge | |
| 4 | Cell Bind, Enhanced | Corning | CellBind | Polystyrene | 125-212 |
| | Attachment 3779 | | | | |

### Optimal Cell Density

The effect of seeding cell densities on the expansion of RPE was done in studies EXP27C, EXP27D, EXP27F and EXP27H

TABLE 3
Results summary of RPE cells seeding in various densities on Star-Plus MC

| | Cell seeding density | | | | | |
|---|---|---|---|---|---|---|
| | 90,000/cm² | | 100,000/cm² | | 120,000/cm² | |
| Study Name | Final Cells/cm² | Yield* | density Cells/cm² | Yield | density Cells/cm² | Yield |
| EXP27C | 1.39E+08 | 8.8 | ND | ND | 1.39E+08 | 6.6 |
| EXP27C | | | ND | ND | 1.45E+08 | 6.9 |
| EXP27C re-plate | 9.77E+07 | 6.2 | ND | ND | ND | ND |
| EXP27D | ND | ND | 1.10E+08 | 6.3 | ND | ND |
| EXP27F | ND | ND | ND | ND | 1.06E+08 | 5.0 |
| EXP27F | ND | ND | ND | ND | 9.20E+07 | 4.4 |
| EXP27F | ND | ND | ND | ND | 1.18E+08 | 5.6 |
| EXP27F | ND | ND | ND | ND | 1.24E+08 | 5.9 |
| EXP27H | ND | ND | 4.03E+07 | 2.3 | ND | ND |
| EXP27J | ND | ND | 9.08E+07 | 5.2 | ND | ND |
| EXP27J | ND | ND | 1.02E+08 | 5.8 | ND | ND |
| EXP27L | ND | ND | | | 7.14E+07 | 3.4 |
| Average | 1.18E+08 | 7.5 | 8.58E+07 | 4.9 | 1.14E+08 | 5.4 |
| SD | 2.90E+07 | 1.8 | 3.13E+07 | 1.8 | 2.60E+07 | 1.2 |
| N | 2 | 2 | 4 | 4 | 7 | 7 |

*Yield was calculated by dividing the number of harvested cells on the last day of passage by the number of seeded cell at day zero of passage.

Conclusion: RPE cells grow in all tested seeding densities as efficiently on MC as on tissue culture flasks.
Fed Bacth vs. ½ media exchange

The effect of various Feeding regimens on RPE expansion on MC was tested in studies EXP27C, EXP27D, EXP27F and EXP27H

TABLE 4
Results summary of testing two feeding regimens along RPE cells
expansion in spinner flasks for optimizing feeding process

| | | Averaged | | Total | | |
|---|---|---|---|---|---|---|
| Study | Condition | Yield | SD | Cells/cm² | SD | N |
| EXP27C | mock 12 B T75 to P4 SF ½ | 6.6 | | 7.94E+05 | | |
| | EX | | | | | |
| EXP27C | mock 12 B SF to P5 SF ½ | 6.9 | | 8.29E+05 | | |
| | EX | | | | | |
| EXP27D | mock 14 H P1 SF | 6.3 | | 4.89E+05 | | |
| EXP27F | mock 14 H SF to P2 SF ½ | 4.4 | | 5.26E+05 | | |
| | EX | | | | | |
| | ½ med ex | 6.05 | 1.13 | 6.59E+05 | 1.77E+05 | 4 |
| EXP27F | mock 14 H SF to P2 SF Fed | 5.9 | | 7.09E+05 | | |
| | Batch | | | | | |
| EXP27J | Mock 14 H ICB P0 to P1 SF − | 3.8 | | 5.26E+05 | | |
| | Nic | | | | | |
| EXP27J | Mock 14 H ICB P0 to P1 | 5.5 | | 6.24E+05 | | |
| | SF + Nic | | | | | |
| EXP27J | Mock 14 H SF P1 to P2 SF − | 5.2 | | 6.23E+05 | | |
| | Nic | | | | | |
| EXP27J | Mock 14 H SF P1 to P2 | 6.0 | | 7.14E+05 | | |
| | SF + Nic | | | | | |
| EXP27H | mock 14 H ICB P0 to P1 SF | 4.7 | | 5.65E+05 | | |
| | Fed Batch | | | | | |
| EXP27H | mock 14 H SF P1 to P2 SF | 7.5 | | 5.97E+05 | | |
| | Fed Batch | | | | | |
| | Fed Batch | 5.5 | 1.15 | 6.23E+05 | 6.98E+04 | 7 |

| | | | | | |
|---|---|---|---|---|---|
| ½ med ex | 6.1 | 1.1 | 6.59E+05 | 1.77E+05 | 4 |
| Fed Batch | 5.5 | 1.2 | 6.23E+05 | 6.98+04 | 7 |

TABLE 5
Recovery Purity and Potency (TEER, and secreted PEDF and
VEGF) post thaw were tested in studies REC#3, RM119

| | | Post thaw % | Purity | TEER | PEDF | VEGF |
|---|---|---|---|---|---|---|
| | | Recovery | (CRALBP/PMEL17) | (Ohm*cm²) | (ng/ml/day) | (ng/ml/day) |
| EXP27F MC | ½ med | 95 | 98.58 | 363 | 4.2 | 3.3 |
| 1A | ex | | | | | |
| EXP27F MC | Fed | 94 | 98.4 | 419 | 4.6 | 2.7 |
| 2A | Batch | | | | | |

Conclusions—Results showed similar values for the two feeding methods. Fed batch was chosen as it is simpler for scale up.

### MC Concentration

The effect of various MC concentration at seeding on RPE expansion was tested in studies EXP27H, and EXP27L, along P2.

TABLE 6
Testing MC concentration. P2 RPE cells were seeded at cell density of
~56.000 cells/cm2 (EXP27H) or ~88,000 cells/cm² (EXP27L)

| | Condition | | | | | | | | |
|---|---|---|---|---|---|---|---|---|---|
| | 5 cm²/mL | | | 10 cm²/mL | | | 20 cm²/mL | | |
| | MC concentration | | | MC concentration | | | MC concentration | | |
| | | Cells | | | Cells | | | Cells | |
| | Total | (10⁶)/ | | Total | (10⁶)/ | | density | (10⁶)/ | |
| Study Name | Cells/cm2 | vessel | Yield | Cells/cm2 | vessel | Yield | Cells/cm2 | vessel | Yield |
| EXP27HP2 − NIC | 5.97E+05 | 104.5 | 7.5 | 4.46E+05 | 156 | 5.6 | 1.47E+05 | 103 | 1.8 |
| EXP27LP2 − NIC | 4.03E+05 | 71 | 3.4 | 4.00E+05 | 140 | 3.3 | | | |
| EXP27HP2 + NIC | 8.49E+05 | 148.5 | 7.1 | 8.86E+05 | 310 | 7.4 | 4.90E+05 | 343.2 | 4.1 |
| EXP27LP2 + NIC | 6.32E+05 | 111 | 5.3 | 5.00E+05 | 175 | 4.2 | | | |
| Average | 6.20E+05 | 108.8 | 5.8 | 5.58E+05 | 195.3 | 5.1 | 3.19E+05 | 223.1 | 3.0 |
| SD | 1.83E+05 | 31.8 | 1.9 | 2.22E+05 | 77.8 | 1.8 | 2.43E+05 | 169.8 | 1.6 |
| N | 4 | 4 | 2 | 4 | 4 | 4 | 2 | 2 | 2 |

### Conclusions:

1. The MC concentration was optimal at the range of 5-10 cm²/mL. The MC concentration that was determined as optimal was 7.2 cm²/mL, i.e. 1 gram of MC (360 cm²) in 50 mL inoculation medium in spinner flask.
2. The most efficient MC concentration is 10 cm²/mL as it enable the highest cells density at end of passage. Even though at 20 cm²/mL twice as many cells are seeded, the final yield is very close to the one achieved by half the MC concentration.

### Reduced Serum in Inoculation Media

TABLE 7
Testing reduced

| | | Condition | | | | | | | | |
|---|---|---|---|---|---|---|---|---|---|---|
| | | 20% HS DMEM | | | 2% HS/Nut(−)HSA | | | Nut(−)HSA | | |
| | | Total | Cells | | Total | Cells | | | Cells | |
| | | Cells/ | (10⁶)/ | | Cells/ | (10⁶)/ | | density | (10⁶)/ | |
| Study Name | Nic | cm2 | vessel | Yield | cm2 | vessel | Yield | Cells/cm2 | vessel | Yield |
| EXP27J | − | 3.63E+05 | 63.5 | 3.8 | NA | NA | NA | NA | NA | NA |
| Mock14H | | | | | | | | | | |
| ICB P1 | | | | | | | | | | |
| EXP27J | − | 6.23E+05 | 109 | 5.19 | NA | NA | NA | NA | NA | NA |
| Mock14H | | | | | | | | | | |
| ICB P2 | | | | | | | | | | |
| EXP27G | + | 2.93E+05 | 51 | 2.4 | NA | NA | NA | 1.80E+04 | 3.2 | 0.2 |
| Mock14O | | | | | | | | | | |
| P3 | | | | | | | | | | |
| EXP27J | + | 5.26E+05 | 92.0 | 5.5 | NA | NA | NA | NA | NA | NA |
| Mock14H | | | | | | | | | | |
| ICB P1 | | | | | | | | | | |
| EXP27J | + | 7.14E+05 | 125 | 5.95 | 8.06E+05 | 141 | 6.71 | NA | NA | NA |
| Mock14H | | | | | | | | | | |
| ICB P2 | | | | | | | | | | |
| EXP29D | + | NA | NA | NA | 4.89E+05 | 85.5 | 4.1 | NA | NA | NA |
| Mock 14 H | | | | | | | | | | |
| ICB P1 | | | | | | | | | | |
| EXP29D | + | NA | NA | NA | 5.29E+05 | 92.5 | 4.4 | NA | NA | NA |
| Mock 14 H | | | | | | | | | | |
| ICB P2 | | | | | | | | | | |

| | | | | | | | | | |
|---|---|---|---|---|---|---|---|---|---|
| Average | 5.04E+05 | 88.2 | 4.6 | 6.08E+05 | 106.3 | 5.1 | 1.80E+04 | 3.2 | 0.2 |
| SD | 1.75E+05 | 30.7 | 1.4 | 1.73E+05 | 30.2 | 1.4 | NA | NA | NA |
| N | 5 | 5 | 5 | 3 | 3 | 3 | 1 | 1 | 1 |

Conclusions—Similar to other studies conducted in TC flasks, the 2% HS concentration in Nut/HSA can replace the standard 20% HS/DMEM seeding media, as it enables the higher cells density at end of passage leading to higher yield and total cells per vessel.

TABLE 8
The presence of Nic along expansion on MC

| | | No Nic | | | | | | | % |
|---|---|---|---|---|---|---|---|---|---|
| | | | Total | | With Nic | | | | increase |
| | initial | final | cells | | initial | final | Total cells | | in yield |
| Study | cell/cm² | cell/cm² | M/Vessel | Yield | cell/cm² | cell/cm² | M/Vessel | Yield | with Nic |
| EXP27H Mock | 9.75E+04 | 5.65E+05 | 98.9 | 4.7 | 9.75E+04 | 9.60E+05 | 168.0 | 8.0 | 70 |
| 14 H ICB P1 | | | | | | | | | |
| EXP27J Mock | 9.60E+04 | 3.63E+05 | 63.5 | 3.8 | 9.60E+04 | 5.26E+05 | 92.0 | 5.5 | 45 |
| 14 H ICB P1 | | | | | | | | | |
| EXP27K Mock | 9.75E+04 | 4.60E+05 | 80.5 | 3.8 | 9.75E+04 | 6.74E+05 | 118.0 | 5.6 | 47 |
| 14 H ICB P1 | | | | | | | | | |
| EXP27J Mock | 1.00E+05 | 6.23E+05 | 109 | 5.2 | 1.00E+05 | 7.14E+05 | 125.0 | 6.0 | 15 |
| 14 H ICB P2 | | | | | | | | | |
| EXP27K Mock | 9.75E+04 | 5.29E+05 | 92.5 | 4.4 | 9.75E+04 | 8.29E+05 | 145.0 | 6.9 | 57 |
| 14 H ICB P2 | | | | | | | | | |
| EXP27L FF | 1.20E+05 | 4.03E+05 | 70.6 | 3.4 | 1.20E+05 | 6.32E+05 | 110.6 | 5.3 | 57 |
| Mock P2 | | | | | | | | | |
| Average | 1.01E+05 | 4.90E+05 | 85.8333 | 4.2 | 1.01E+05 | 7.22E+05 | 126.4 | 6.2 | 48.2 |
| SD | 9.19E+03 | 9.93E+04 | 17.4 | 0.7 | 9.19E+03 | 1.53E+05 | 26.8 | 1.1 | 18.7 |
| N | 6 | 6 | 6 | 6 | 6 | 6 | 6 | 6 | 6 |

| | | | | |
|---|---|---|---|---|
| TTEST No Nic Vs. with Nic | 1.00 | 0.0131 | 0.0131 | 0.004 |

Conclusions: Addition of Nic resulted in 50% increase in final cell yield of each passage. Results were significant (P<0.05)

Conclusions for the development phase of RPE expansion on me in 0.1 L spinner flasks:
1. From the six types of MCs tested, the Star-Plus MCs were found to best support RPE growth.
2. RPE cells grew well in the range of 90,000 cell/cm² to 120,000 cells/cm². This range of densities is identical to the range found on TC flasks.
3. RPE growth was comparable with the two feeding methods. Fed batch was chosen over ½ media exchange as it is simpler for scale up when using the MC and it is more cost effective.
4. The most efficient MC concentration was found to be 10 cm²/mL as it enables the highest cells density at end of passage. Even though at 20 cm²/mL, twice as many cells were seeded, the final yield was very close to the one achieved by half the MC concentration.
5. Similar to previous studies conducted in TC flasks, the 2% HS concentration in nut/HSA can replace the standard 20% HS/DMEM seeding media as it enable the higher cells density at end of passage. The yield and total cells per vessel were higher as well.
6. Addition of Nic resulted in 50% increase in final cell yield of each passage. Results were significant (p<0.05)

### Example 3

### Screening for Additional Suitable MC Types

In RPE-Pro-05 study additional MC type were screened for their suitability to support RPE expansion. The commercially available microcarriers tested in this study are shown in Table 9

TABLE 9
Additional testing of MC suitability for RPE expansion

| | | | | | Diameter |
|---|---|---|---|---|---|
| | MC Type and Cat# | Company | Charge/Coating | Matrix | (μM) |
| 1 | Solohill Star-Plus SP102-1521 | PALL | Positive (electric) | Crosslinked | 125-212 |
| | | | | Polystyrene | |
| 2 | Solohill Hillex ®II H112-170 | PALL | Positive (chemical) | Modified | 160-200 |
| | | | | polystyrene | |
| 3 | Solohill Plastic Plus PP102- | PALL | Positive | Cross-linked | 125-212 |
| | 1521 | | | polystyrene, | |
| | | | | cationic charge | |
| 4 | Synthemax II Low | Corning | Synthemax ® | Polystyrene | 125-212 |
| | Concentration 3781 | | | | |
| 5 | Synthemax II High | Corning | Synthemax ® | Polystyrene | 125-212 |
| | Concentration 3784 | | | | |
| 6 | Cell Bind, Enhanced | Corning | CellBind** | Polystyrene | 125-212 |
| | Attachment 3779 | | | | |

### Example 4

The purpose of this example is to summarize the development of a scaled-up process for the expansion of RPE cells in Eppendorf's single use 3 L bioreactor (SUB) BioBlu 3C, monitored by Eppendorf's BioFlo 320 bioprocess system.

The development of RPE expansion on Star Plus microcarriers (MC) in spinner flasks provided the basis for process parameters that can be applied further in preliminary studies to expand RPE cells on microcarriers (MC) under controlled conditions in a bioreactor. These parameters are seeding cell density, seeding agitation speed, MC concentration, and feeding regime.

This system utilizes its proprietary software to monitor and control cell culture applications that demand varying and continuous control of process parameters required in cell culturing, including aeration regime (Air, O₂, CO₂, N₂) pH, temperature and agitation speed.

Utilizing a biowelder for connecting media-containing bags allowed the SUB to be maintained as a ‘closed-system’. The current filtration procedure for separating RPE cells from MC and other large particles (matrix and cell aggregates) which was previously conducted manually using an open 40 micron mesh, was replaced, aiming to sustain a closed-system which is able to support significant scale up.

### Materials, Equipment and Cells

TABLE 10
Materials

| Item | Manufacturer | Cat. No. |
|---|---|---|
| 2 L/2 ports bag | Meissner | MEIDUF3102B- |
| | | N00-B8304-01 |
| 2 L/3 ports bag | Meissner | MEIDUF4102B- |
| | | N00-B8304-01 |
| 5 L bag | Meissner | MEIDUF2104B- |
| | | N00-B9126-01 |
| 45% (w/v) D-glucose solution | Sigma-Aldrich | G8769 |
| 50 μm filter | Sartorius | 5055350P9FF |
| 60 μm filter | Meissner | CL2MN60772 |
| Bioblu 3C SUB | Eppendorf | 1386000300 |
| DMEM | Biological Industries | SH30081.01 |
| Glucose measurement strips | Roche | 11447475 |
| HS (human serum) | Akron | AK99050100 |
| Lactate measurement strips | Roche | 03012654 |
| L-Glutamine | Hyclone | SH30034.01 |
| MC | PALL | AMDS05SPS100 |
| NUT (−) + has | Biological Industries | 065100011A |
| P/S | Biological Industries | 030311B |
| PBS (−) | Hyclone | SH30028.02 |
| Tryple Select | Gibco | 12563029 |
| Via-1 Cassette | Chemometec | 9410011 |

TABLE 11
Equipment

| Equipment | CCN ID# |
|---|---|
| Accutrend Plus metabolite tester | 600-AP-002 |
| Clipster | 372-CL-002 |
| Eppendorf's BioFlo 320 bioprocess system | 530-BR-001 |
| NC-200 | 135-NC-005 |
| Sartorius Biowelder TC | 540-BW-001 |

### Abbreviations and Definitions

CS5—Cryostor 5%
DP—Drug Product.
FFMD—feeder free monolayer differentiation
HSA—Human Serum Albumin
ICB—intermediate cell bank
MC—microcarriers
Nic—Nicotinamide
NUTS—NutriStem (cell culture media)
OpRegen® TAI—OpRegen® ‘Thaw-and-Inject’
RPE—Retinal Pigmented Epithelial cells.
RPM—rounds per minute
SF—spinner flask
SUB—Single use bioreactor

### Experimental Design

RPE cells were expanded on T175 flasks from P0 (end of RPE cell differentiation and start of expansion) until the end of the first passage, P1, 9-14 days. Cells were then harvested and the SUB was inoculated at a cell density of 90,000-120,000 cells/cm² with a MC concentration of 3.6-7.2 cm²/mL. 3 to 4 days post-inoculation, media (20% HS/DMEM) was replaced with growth media Nutristem minus (Nut (−)) with or without HSA. Glucose and lactate levels were measured prior to media addition and Glucose was supplemented to 2-3 grams/L (plus additional glucose to compensate weekends according to current consumption).

As control for expansion on MC, approx. 10 min post-inoculation cells were sampled from the SUB and transferred to a spinner flask which culture in parallel. Other process modifications were tested and implemented during each run and are documented in Table 12.

TABLE 12
Study design list in the order of execution

| Study #/ | Parameter | | | Comments/ |
|---|---|---|---|---|
| cell origin | tested | description | Readouts | Conclusions |
| MCS1 | Nut HSA + Nic | Initial inoculation | P6 Day 3 | Cell did not attach well, |
| EXP27L P5 | Agitation 40 | agitation was set | Attachment & | only ~20%. Seeding |
| SFx5 | RPM for all the | to 40 RPM | Morphology by | agitation was too high. |
| | expansion | | microscope | 22 RPM is the equivalent |
| | Harvest day 5 | | Counting unbound cells. | speed to the 40 RPM of |
| | | | Yield day 5 was 3.5 by | spinner flask. Seeding |
| | | | standard filtration. | medium has high pH |
| | | | Day 17 (day 5 MC | (7.7, pH levels should be |
| | | | transferred from SUB to | optimized in the future. |
| | | | SF) yield was 3.7 | Relative to the pH of |
| | | | | growth medium of 7.3). |
| | | | | Cultured up to day 5 in |
| | | | | the SUB, RPE grew well |
| | | | | up to discontinuation of |
| | | | | the SUB. |
| | Filtration | Enzyme was | Yield day 5 was 0.7 | Cultured up to day 5 in |
| | Sartopure Vs. | quenched with | after Sartopure | the SUB. Filtration |
| | standard 40- | Nut (−). Sartopure | | should be improved- |
| | micron mesh | filter was washed | | reducing filtration speed. |
| | | with PBS before | | Filter should be pre- |
| | | use. | | wetted with quench |
| | | | | solution containing |
| | | | | serum/serum albumin. |
| MCS2/ | Nut HSA | Passaging RPE | P7 Day 3 Morphology | Cells attached and grew |
| MCS1 P6 | Seeding | cells from SUB to | Yield (Day 3 MC | well, ~28%. |
| day 17 | Agitation 22 | SUB. High | transferred from SUB to | P7 Cells reached |
| | RPM | passage because | SF) harvested at day | senescence as expected. |
| | Filtration: | no other cell | 14 = 0.7, in Control | SUB was aborted on day |
| | standard 40- | source was | SF = 1.2 | 3. |
| | micron mesh | available | | |
| MCS3/ | Nut HSA + | Small number | P3 | RPE grew well in SUB |
| Live HESPRO04 | Nicotinamde | of cells. only | Day 9 yield was 4 (3 | Filtration with the |
| GRPD P2 | (Nic) | 222M were | mL sample) | Sartopure was |
| | Seeding | seeded in 1.62 | Day 11 Yield (Table | successful |
| | Agitation 22 | cm²/mL MC | 5) | |
| | RPM then | concentration, | Morphology | |
| | 29 RPM | 91,000 cells/ | Yield was 8.5 before | |
| | seeding | cm2 | filtration, after | |
| | densities | | Filtration Sartopure | |
| | Feeding | | (6.8) was similar to | |
| | Filtration | | standard 40 um (7.0) | |
| | Sartopure Vs. | | | |
| | standard 40- | | | |
| | micron mesh | | | |
| MCS4/HESPRO04 | LM521 coating | Testing | Aborted | Faulty heating blanket |
| GRPA P0 | | expansion at P1 | | |
| | | on LM521 | | |
| | | before seeding | | |
| | | on MC in SUB | | |
| MCS5/HESPRO04 | LM521 coating | Testing | P2 Day 14 Yield | Aborted on day 5. Cells |
| GRPG P0 | | expansion at P1 | (Table 5) | did not attach well to |
| | | on LM521 | Morphology | the MC in SF and SUB. |
| | | before seeding | Recovery | Fell off from MC |
| | | P2 on MC in | | sample drawn by |
| | | SUB | | syringe from SUB. |
| MCS6/FF run#4 | MC | A small number | P2 Day 17 Yield | Cells grew well in SUB |
| end of P2 grown | concentration × | of cells were | (Table 5) | and SF (yield = 5) but |
| on Gelatin | 0.5 | inoculated. only | Morphology | were lost in filtration |
| | Filtration: | 373 × 10⁶ were | Recovery, purity | (Sartopure) because |
| | Sartopure Vs. | seeded on MC | potency | filter was washed in |
| | standard 40- | (in | | DMEM and not |
| | micron mesh | concentration of | | Nutristem HSA. |
| | From SF: | 2 cm²/mL) | | Filtration with 40 um |
| | Control 40 um | Inoculation | | control and Vanguard |
| | Vanguard | density was | | (Meissner) 60 um (from |
| | 60 um | 120,000 cells/ | | SF) was successful. |
| | Vanguard | cm² | | |
| | 30 um | | | |
| MCS7/Thaw 5 | Thaw ICB | P2 on gelatin- | P3 Day 10,13,14,16 | P3 Yield was high (5.5- |
| vials of | Nic | coated flask for | Yields | 8.5) and increased from |
| HESPRO04 | DO (dissolved | adaptation | Morphology | day 10 to 16. |
| GRPA LM521 | oxygen) | before seeding | Recovery, purity | Filtration in Sartopure |
| P1 CS5 | Yield and potency | P3 in SUB with | potency, follow-up P4 | succeeded. |
| 50M/vial | along passage | Nut HSA + Nic | in T25 morphology | Follow-up P4 |
| | | Changed DO | | expansion in T25 and |
| | | from 20% to | | direct potency show |
| | | 30% | | optimal harvest day was |
| | | | | day 14 |
| MCS8/CCN003 | GMP P1 RPE | DO electrode | P2 Day 13,14 Yields | P2 Yield was low (1- |
| P1 | from ongoing | was disabled, | Recovery, purity | 1.9) probably the flow |
| | production. | sensor cable was | potency, follow-up P4 | of air/oxygen was too |
| | Nut (−) | torn. O₂ flow | in T25 morphology | low and impacted cell |
| | Sartopure Vs. | was set manually | | growth. |
| | standard 40- | to 0.002 L/min | | 30% loss in filtration. |
| | micron mesh | and Air to 0.02 | | Probably due to small |
| | Cryopreservation: | L/min | | number of cells relative |
| | Filling with the | | | to the amount of MC. |
| | Fill it machine | | | Cells stuck to the MC |
| | Vs. Manual, 1M, | | | during filtration. |
| | 2M, 5M per vial | | | |

### Experimental Procedure

Thawed or ongoing sources of differentiated RPE cells were derived from hESC grown on human cord or in feeder free conditions, were expanded in T-flasks before seeding on MC in SUB. Cells were counted using the NC-200 cell counter and yield was calculated by dividing by the number of inoculated cells. Cell morphology and lactate and glucose levels were examined along passage. At end of the passage RPE cell cultures of each condition were harvested and yield was compared to the control spinner flask (cultured with RPE cells that were sampled 10 minutes after SUB inoculation with 1/30 (50 ml) of the SUB inoculation volume). In some experiments, frozen cell samples at end of passage were tested for recovery, identity and potency

TABLE 13
Cells used in the development

| | | | Date of | |
|---|---|---|---|---|
| Cells | Passage | Bank | cryopreservation | Related study |
| Live FF | P5 | EXP27L P5 SFx5 | NA | MCS1 + MCS2 |
| RPE | | | | |
| Live FF | P2 | HESPRO04 GRPD | NA | MCS3 |
| RPE | | | | |
| Live FF | P1 | HESPRO04 GRPA | NA | MCS4 + MCS5 |
| RPE | | | | |
| Live FF | P2 | FF run#4 | NA | MCS6 |
| RPE | | | | |
| Frozen FF | P1 | HESPRO04 GRPA LM521 | Dec. 13, 2019 | MCS7 |
| RPE | | 50M/v | | |
| Live | P1 | CCN003 | NA | MCS8 |
| OpRegen ® | | | | |

### Results

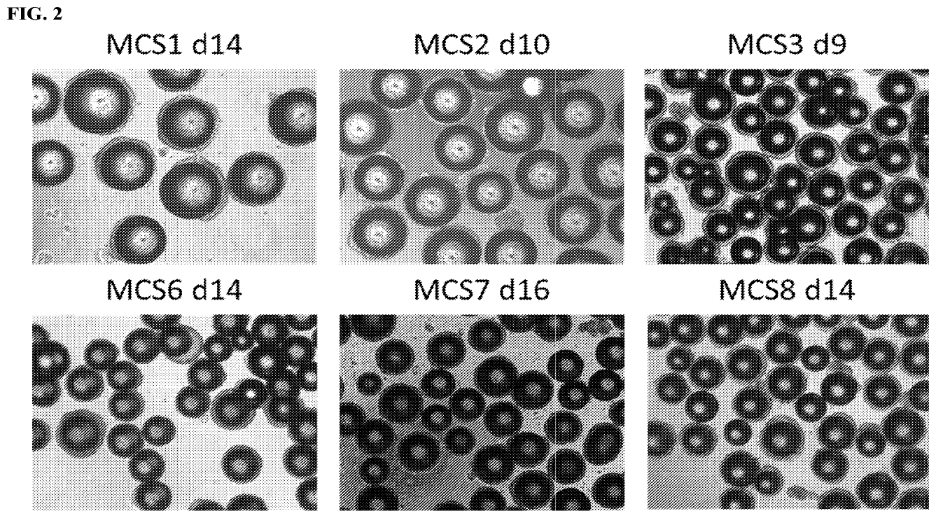

The main findings are summarized below. Morphology is shown in FIG. 2. Morphological assessment of indicated MCS studies shows the outer layer of RPE cells on MCs' surface. Representative phase images with 4× objective of RPE cells attached to MC near the end of passage (day number is indicated in FIG. 2).

### Yield

TABLE 14
Cell counts and related parameters at inoculation and final yield estimations

| | seeding | | | | | | | | | | |
|---|---|---|---|---|---|---|---|---|---|---|---|
| | | final | | | | | | | | | |
| | | surface | initial | | Harvest | | | | | | |
| | | Density | Cell | Total | Last | cell/cm² | cell/cm² | cell/cm² | Total | | |
| | area | cm²/ | Density/ | cells | day of | pre | post | pre | cells | Final | |
| Study | cm² | ml | cm² | seeded | passage | filter | filter | cryo | M/Vessel | Yield | notes |
| MCS1 | 12,000 | 8.0 | 1.05E+05 | 1.26E+09 | 17 | NA | NA | 3.83E+05 | 4.60E+09 | 3.7 | *5 d |
| | | | | | | | | | | | (SUB)17 |
| | | | | | | | | | | | d(SF) |
| MCS2 | 4,800 | 3.2 | 1.25E+05 | 6.00E+08 | 14 | NA | NA | 8.29E+04 | 435000000 | 0.73 | *3 d |
| | | | | | | | | | | | (SUB)14 |
| | | | | | | | | | | | d(SF) |
| MCS3 | 2,430 | 1.6 | 9.10E+04 | 2.21E+08 | 11 | 7.41E+05 | 6.42E+05 | 8.54E+05 | 2.08E+09 | 9.38 | NA |
| MCS6 | 3,096 | 2.1 | 1.20E+05 | 3.72E+08 | 17 | 6.27E+05 | 1.89E+05 | 1.37E+05 | 4.09E+08 | 1.14 | NA |
| MCS7 | 9,724 | 7.2 | 1.15E+05 | 1.24E+09 | 16 | 8.88E+05 | 1.06E+06 | 1.06E+06 | 1.04E+10 | 8.35 | ** Final |
| | | | | | | | | | | | MC area |
| MCS8 | 7829 | 5.5 | 1.09E+05 | 8.95E+08 | 14 | 2.71E+05 | 1.40E+05 | 1.40E+05 | 1.09E+09 | 1.22 | **Final |
| | | | | | | | | | | | MC area |

*In MCS1 and MCS2 culture was transferred into spinner flasks. Final indicated yields are derived from harvested spinner flasks.
**In MCS7 and MCS8 several samples were taken (each of 3% of the total volume) along passage. MC surface area shown in Table 14 (column 3 - final surface density) is final at harvest; Inoculation MC surface areas were MCS7- 10,800 cm², MCS8- 8,208 cm².

### Glucose and Lactate Measurements

TABLE 15
Glucose consumption rate along passage
Glucose consumed nmol/mL/day

| Days in | MCS3 | | MCS6 | | MCS7 | | MCS8 | |
|---|---|---|---|---|---|---|---|---|
| culture | SUB | SF | SUB | SF | SUB | SF | SUB | SF |
| 2 | 1.4 | 4.9 | NA | NA | 2.9 | 2.7 | NA | NA |
| 3 | 1.0 | 5.1 | 1.2 | 1.0 | 2.9 | 3.2 | 2.9 | NA |
| 4 | 2.3 | 2.6 | 1.5 | 0.7 | NA | NA | 2.6 | 0.2 |
| 6 | NA | NA | NA | NA | 3.2 | 3.3 | 5.8 | 4.9 |
| 7 | 1.9 | 2.5 | 1.7 | 1.0 | 3.9 | 2.2 | 2.2 | 1.8 |
| 8 | NA | NA | NA | NA | 4.4 | 0.0 | NA | NA |
| 9 | 4.9 | 6.1 | NA | NA | 4.2 | 1.7 | NA | NA |
| 10 | NA | NA | 1.2 | 0.7 | 1.5 | 1.6 | 1.2 | 1.9 |
| 12 | NA | NA | NA | NA | NA | NA | 2.4 | 0.3 |
| 13 | NA | NA | NA | NA | 2.1 | 0.0 | 0.6 | NA |
| 14 | NA | NA | 1.0 | 0.7 | NA | NA | 0.4 | 1.4 |

TABLE 16
Lactate consumption rate along passage
Lactate produced nmol/mL/day

| Days in | MCS3 | | MCS6 | | MCS7 | | MCS8 | |
|---|---|---|---|---|---|---|---|---|
| culture | SUB | SF | SUB | SF | SUB | SF | SUB | SF |
| 2 | 1.8 | 10.3 | NA | NA | 5.0 | 4.8 | NA | NA |
| 3 | 2.3 | 8.7 | 2.4 | 1.9 | 7.4 | 8.0 | 6.5 | 0.0 |
| 4 | 2.6 | 2.9 | 2.9 | 2.0 | NA | NA | 3.4 | 5.5 |
| 6 | NA | NA | NA | NA | 5.2 | 4.7 | 11.1 | 6.7 |
| 7 | 3.6 | 1.8 | 3.0 | 0.0 | 5.5 | 0.0 | 1.5 | 4.9 |
| 8 | NA | NA | NA | NA | 7.1 | 7.8 | NA | NA |
| 9 | 6.6 | 0.0 | NA | NA | 0.0 | 0.0 | NA | NA |
| 10 | NA | NA | 10.0 | 0.6 | 0.0 | 0.0 | 1.9 | 0.0 |
| 12 | NA | NA | NA | NA | NA | NA | 2.9 | 2.0 |
| 13 | NA | NA | NA | NA | 0.0 | 0.0 | 6.4 | 0.0 |
| 14 | NA | NA | 2.1 | 0.5 | NA | NA | 1.5 | 1.4 |

Glucose consumption and lactate production were calculated by subtracting the previous level (nmol/mL) from the current one, and dividing result by the duration (no. of days) since previous measurement. While in most cell lines that are cultured in bioreactors, such as CHO cells, lactate production level constantly increases, in RPE cell culture it does not. A relative decrease in glucose consumption indicated that the culture reached confluency, while a decrease in Lactate production indicate a shift from glycolysis to Oxidative phosphorylation with possible lactate consumption by the RPE cells.

TABLE 17
TEER measurements (Modified assay results as per QC-WIN-11)

| | TEER |
|---|---|
| Study | (Ohm*cm²) |
| MCS1 d17 | NA |
| MCS2 d14 | NA |
| MCS3 d11 | 0.48 |
| MCS6 d17 | NA |
| MCS7 d14 | 236 |
| MCS7 d16 | 35 |
| MCS8 d14 | 293 |

### Conclusions

The BioBlu 3C SUB combined with the BioFlo 320 bioprocess system have demonstrated repeatability and robustness in maintaining and supporting the expansion of RPE cells on MC under controlled and monitored environment. The 3-gas mix algorithm (O₂, CO₂, and air) is efficient in maintaining RPE growth at 30% DO set point. Agitation was tuned along process; in order to achieve adequate cell attachment inoculation was executed at 22 RPM, then agitation was further increased to 29 RPM for full suspension of MC, and agitation was finally elevated to 40 RPM to increase the efficiency of gas dissolution in the culture to facilitate the increased oxygen consumption rate by the expanding cells. Four independent runs (MCS3, MCS6, MCS7, and MCS8) gave good cell yields at the end of expansion in the BioBlu 3C SUB before filtration. Filtration using the Sartopure 50 μm filter was successful in 2 runs while in 2 other runs significant loss of cells was observed. Efforts to further improve the filtration process are ongoing. In 2 out of 3 runs, thawed vials passed the potency assay, indicating that RPE cells that were expanded in the SUB on MC retained their properties and biological activity. Optimal harvest day was found to be Day 14, hence future cell harvesting will be executed on days 13-14 to allow operational flexibility.

Final process parameters are summarized in Table 18.

TABLE 18
Solution volumes, MC amount and process parameters setpoints

| | | |
|---|---|---|
| # | Parameter | Value/SP |

Cells per Inoculation

| | | |
|---|---|---|
| 1 | Target cell density (cells/cm²) | 50K-200K |
| 2 | Total inoculation cell No. (×10⁶) for a final conc. of 100,000- | 500-2,000 |
| | 120,000 cells/cm² | |
| 3 | Total inoculation cell suspension volume (mL) (4.2-5 × 10⁶ cells/mL) | 100-500 |

MC Details

| | | |
|---|---|---|
| 4 | Amount per BioBlu 3C (gr.) | 10-60 |
| 5 | MC concentration (cm²/ml) | 2-10 |
| 6 | Total surface per SUB (cm²) | 8,000-15,000 |

Inoculation media ingredients

| | | |
|---|---|---|
| 7 | Volume of MC in DMEM suspension per BioBlu 3C (mL) | 400-1000 |
| 8 | Supplementary DMEM volume (to a final 1500 mL) | 100-500 |
| 9 | Human Serum (HS) (mL) | ~100-500 |
| | | (~6%-34%) |
| 10 | L-Glutamine (mL) | 5-30 (0.3-2%) |
| 11 | Pen-Strep (P/S) (mL) | 1-10 |
| 12 | Total inoculation volume (mL) | 500-5000 |

Fed Batch media

| | | |
|---|---|---|
| 13 | Inoculation media replacement solution NUT (−) with HSA (ml) | 500-5000 |
| 14 | Fed batch media NUT (−) with HSA (ml., each) | 100-1000 |
| 15 | Glucose 45% supplement to 2.5 g/L (plus extra consumption day on | When below 2 |
| | weekend) | g/L |

Process Parameters

| | | |
|---|---|---|
| 16 | Agitation (rpm) | 10-30 (d0), |
| | | 10-40 (d4), |
| | | 20-50 (d7) |
| 17 | Temp. | 37° C. |
| 18 | DO | ≥30% |
| 19 | pH | 7.25 ± 1.25 |
| 20 | Sparger (SLPM) | 0.002-0.050 |
| 21 | Overlay (SLPM) | 0.0-1.0 |

TABLE 19
Data Summary
Expansion in SUB

| | | | | Study Conditions | | | | | | | | |
|---|---|---|---|---|---|---|---|---|---|---|---|---|
| | Cell Source | | | | | | | Stirring | Post | Post | Beads | |
| | Live/ | | | Target | | area | seeding | scheme for | plating | [symbol] fluence | Density | Seeding Cell |
| Study | Frozen | Cell Batch | Passage | Vessel | Passage | cm2 | medium | plating | RPM | RPM | cm2/ml | Density/cm2 |
| MCS1 | live | EXP27LP6 | 5 | BioBlu3C | 6 | 12,000 | 20% HS | 40 RPM | 30 | 30 | 10 | 1.05E+05 |
| | | | 5 | SF | 6 | 1,800 | 20% HS | 40 RPM | 50 | 50 | 10 | 6.00E+04 |
| | | | 5 | T175 | 6 | 175 | 20% HS | NA | NA | NA | 5.0 | 1.20E+05 |
| MCS2 | live | MCS1 | 5 | BioBlu3C | 7 | 4,800 | 20% HS | 40 RPM | 22 | 22 | 5.0 | 1.25E+05 |
| | | | 5 | SF | 7 | 175 | 20% HS | 40 RPM | 50 | 50 | 5.0 | 1.20E+05 |
| | | | 5 | T175 | 7 | 175 | 20% HS | NA | NA | NA | 5.0 | 1.20E+05 |
| MCS3 | live | HESPRO04 GRPD | 2 | BioBlu3C | 3 | 2,430 | 20% HS | 22 RPM | 29 | 29 | 1.6 | 9.10E+04 |
| | | | 2 | T175 | 3 | 175 | 20% HS | NA | NA | NA | 5.0 | 9.03E+04 |
| MSC4 | live | HESPRO04 GRPA | 1 | BioBlu3C | 2 | | | | | | | |
| MSC5 | live | HESPRO04 GRPD | 1 | BioBlu3C | 2 | 9,072 | 20% HS | 22 RPM | 29 | 29 | 6.0 | 1.02E+05 |
| | | | 1 | SF | 2 | 302 | 20% HS | 40 RPM | 50 | 50 | 6.0 | 1.02E+05 |
| | | | 1 | T25 × 2 | 2 | 50 | 20% HS | NA | NA | NA | NA | 1.20E+05 |
| MSC6 | live | HESPRO04 run4 | 2 | BioBlu3C | 3 | 3,096 | 20% HS | 22 RPM | 29 | 29 | 2.1 | 1.20E+05 |
| | | | | | | 103 | | | | | | 1.20E+05 |
| | | | 2 | SF | 3 | 103 | 20% HS | 40 RPM | 50 | 50 | 2.1 | 1.20E+05 |
| | | | | | | 103 | | | | | | 1.20E+05 |
| MSC7 | ICB | HESPRO04 GRPA | 2 | BioBlu3C | 3 | 232 | 20% HS | 22 RPM | 29 | 40 | 4.6 | 1.15E+05 |
| | | LM521 P1 CS5 | | | | 176 | | | | | | 1.15E+05 |
| | | 50M/via ×5 | | | | 173 | | | | | | 1.15E+05 |
| | | | | | | 138 | | | | | | 1.15E+05 |
| | | | | | | 9,724 | | | | | | 1.15E+05 |
| | | | 2 | SF | | 360 | 20% HS | 40 RPM | 50 | 50 | 7.2 | 1.15E+05 |
| | | | 2 | T25 × 1 | 3 | 25 | 20% HS | NA | NA | NA | 5.0 | 1.15E+05 |
| MSC8 | live | CCN003 P1 | 1 | BioBlu3C | 2 | 137 | 20% HS | 22 RPM | 29 | 40 | 5.5 | 1.20E+05 |
| | | | | | | 137 | | | | | | 1.09E+05 |
| | | | | | | 106 | | | | | | 1.09E+05 |
| | | | | | | 7,829 | | | | | | 1.09E+05 |
| | | | 1 | SF | 2 | 274 | 20% HS | 40 RPM | 50 | 50 | 5.5 | 1.09E+05 |

| | | | | | | Results | | | | |
|---|---|---|---|---|---|---|---|---|---|---|
| | Total cells | Growth | | final [symbol] | | Last day of | days from | | Total cells | Final |
| Study | seeded | Medium | Nic | mL | [symbol] seperation | passage | last feed | final cell cm2 | M/Vessel | Yield |
| MCS1 | 1.26E+09 | Nut | + | 1500 | S [symbol] topure SP9 | 5 | 2 | 8.81E+04 | 881 | 0.84 |
| | 1.08E+08 | Nut | + | 134 | 40 um | 17 | 3 | | 127 | 6.02 |
| | 2.10E+07 | Nut | + | 35 | 40 um | 18 | 2 | 2.97E+05 | 52 | 2.48 |
| MCS2 | 6.00E+08 | Nut | − | 1500 | 40 um | 14 | 3 | 8.29E+04 | 14.5 | 0.66 |
| | 2.10E+07 | Nut | − | 106 | 40 um | 14 | 3 | 1.42E+05 | 24.8 | 1.18 |
| | 2.10E+07 | Nut | − | 35 | 40 um | 14 | 3 | 5.11E+05 | 89.5 | 4.26 |
| MCS3 | 2.21E+08 | Nut HSA | + | 2 [symbol] | S [symbol] topure [symbol] | 11 | 4 | 8.54E+05 | 2.08E+09 | 9.38 |
| | | | | | 40 um | | | | | |
| | 1.58E+07 | Nut HSA | + | 35 | 40 um | 11 | 4 | 6.29E+05 | 110 | 6.96 |
| MSC4 | Aborted due to faulty Heat Jacket | | | | | | | | | |
| MSC5 | 9.24E+08 | Nut HSA | − | 1500 | Cells fully detached from the SUB study aborted | | | | | |
| | 3.08E+07 | | − | 50 | 40 um | 12 | 3 | 3.97E+05 | 120 | 3.90 |
| | 6.00E+06 | | − | 5 | NA | 12 | 3 | 9.76E+05 | 24.4 | 8.13 |
| MSC6 | 3.72E+08 | | + | 3000 | S [symbol] topure | 17 | 3 | 1.37E+05 | 4.09E+08 | 1.14 |
| | 1.24E+07 | | | | 40 um | 17 | 3 | 3.95E+05 | 1.78E+09 | 4.95 |
| | 1.24E+07 | Nut HSA | + | 92 | Vangard 60 um | 17 | 3 | 5.4 [symbol] E+05 | 5.66E+07 | 4.56 |
| | 1.24E+07 | | | | Vangard 30 um | 17 | 3 | 4.31E+05 | 4.45E+07 | 3.59 |
| MSC7 | 2.66E+03 | Nut HSA | + | 50 | 40 um | 10 | 3 | 2.71E+05 | 6.57E+09 | 5.30 |
| | 2.00E+07 | | | 50 | 40 um | 13 | 1 | 7.39E+05 | 7.67E+09 | 6.19 |
| | 1.99E+07 | | | 50 | 40 um | 14 | 3 | 8.38E+05 | 3.41E+09 | 6.78 |
| | 1.59E+07 | | | 50 | 40 um | 16 | 3 | 8.76E+05 | 8.64E+09 | 6.97 |
| | 1.12E+09 | | | 3000 | S [symbol] topure | 16 | 3 | 1.06E+06 | 1.04E+10 | 8.35 |
| | 4.13E+07 | Nut HSA | + | 50 | 40 um | 17 | 4 | 4.49E+05 | 1.62E+08 | 3. [symbol] 1 |
| | 3.00E+06 | Nut HSA | + | 5 | 40 um | 13 | 1 | 9.72E+05 | 2.43E+07 | 8.10 |
| MSC8 | 1.49E+07 | NUT HSA | − | 50 | 40 um | 14 | 2 | 2.33E+05 | 1.88E+09 | 1.52 |
| | 1.49E+07 | | | 50 | 40 um | 14 | 2 | 1.23E+05 | [symbol] .84E+08 | 0. [symbol] 9 |
| | 1.15E+07 | | | 20 | 40 um | | | 2.11E+05 | 1.68E+09 | 1. [symbol] 7 |
| | 854000000 | | | 3000 | S [symbol] topure | | | 1.40E+05 | 1.09E+09 | 1.22 |
| | 2.98E+07 | NUT HSA | − | 50 | 40 um | 14 | 2 | 2.06E+05 | 5.65E+07 | 1.89 |

[symbol]

indicates data missing or illegible when filed

### Example 5

This example summarizes the development of a scaled-up production process for the expansion of RPE cells (for one expansion passage) on MC in a 3 L single-use bioreactor using Eppendorf's Bioflo 320 bioprocess system, based on previously established process development.

OPREGEN® ‘Thaw-and-Inject’ (OPREGEN® TAI) process development includes the implementation of a large-scale production process—encompassing the inoculation, expansion, and harvesting of Retinal Pigmented Epithelial (RPE) cells—under monitored and controlled conditions. The development of this scaled-up process focused on the expansion of RPE cells in their final (P2) expansion phase, with earlier expansion phases (P0 and P1) currently remaining flask-dependent and unchanged from current practice.

The basis of this transition, from a flask-dependent process to a large-scale, semi-automated closed-system relies on two fundamental changes: One is the assimilation of a benchtop bioprocess system—e.g., Eppendorf's BioFlo 320—with the flexibility to control single-use vessels in cell culture processes. The system utilizes its proprietary software to monitor and control cell culture applications that demand varying and continuous control of process parameters required in cell culturing. The second change is the ability to grow the RPE cells on microcarriers (MC) which provide the surface required for adhesion of the RPE cells, replacing the gelatin-coated tissue culture flasks surface without affecting OPREGEN® quality attributes and characteristics. This developed process will also contribute to the overall intention of reducing aseptic risk during the OPREGEN® production process.

### Objectives and Scope

Summarize the development of a scaled-up production process for expansion of RPE cells (one passage of expansion) on MC in a 3 L single-use bioreactor using Eppendorf's Bioflo 320 bioprocess system. Introduce and delineate the OPREGEN® TAI scaled-up production process.

### Abbreviations and Definitions

BioFlo 320—Eppendorfs automated monitoring and control bioprocess system.
BLOD—Below Limit of Detection.
Dead Band—The span of input within a specified range for which there is no change in the output.
DMEM—Dulbecco's Modified Eagle Medium.
DO—Dissolved Oxygen (%).
DS—Drug Substance.
ECM—Extra Cellular Matrix.
Fed Batch—operational technique where one or more nutrients are fed (supplied) to the bioreactor during cultivation and in which the product(s) remain in the bioreactor until the end of the run.
FF—Feeder-Free hESC.
FFMD—Feeder-Free Monolayer Differentiation.
GRP—group
HS—Human Serum.
HSA—Human Serum Albumin.
ICB—Intermediate Cell Bank.
Inoculation—The transfer of cells into a bioreactor.
LM521—Laminin 521.
MC—Microcarriers.
NUT(−)/HSA—NutriStem (cell culture medium) containing HSA.
OpRegen® TAI—OpRegen® ‘Thaw-and-Inject’ (new OpRegen® formulation).
PBS—Phosphate buffered Saline.
RPE—Retinal Pigmented Epithelial cells.
RPM—Revolutions Per Minute.
SF—Spinner Flask.
SLPM—Standard Liter Per Minute.
SP—Set Point.
SUB—Single-Use Bioreactor.
Temp.—Temperature.
w/v—weight to volume ratio.

TABLE 20
Materials and Disposables.

| Item | Manufacturer | Cat. No. |
|---|---|---|
| 2 L/2 ports bag | Meissner | MEIDUF3102B-N00-B8304-01 |
| 2 L/3 ports bag | Meissner | MEIDUF4102B-N00-B8304-01 |
| 45% (w/v) D-glucose solution | Sigma-Aldrich | G8769 |
| 40 μm grid mesh | pluriSelect | 43-50040-01 |
| 50 μm filter | Sartorius | 5055350P9FF |
| 5 L bag | Meissner | MEIDUF2104B-N00-B9126-01 |
| 60 μm filter | Meissner | CL2MN60772 |
| Bioblu 3C SUB | Eppendorf | 1386000300 |
| DMEM | Biological Industries | SH30081.01 |
| Glucose measurement strips | Roche | 11447475 |
| HS | Akron | AK99050100 |
| Lactate measurement strips | Roche | 03012654 |
| L-Glutamine | Hyclone | SH30034.01 |
| MC | PALL | AMDS05SPS100 |
| NUT (−) + HSA | Biological Industries | 065100011A |
| P/S | Biological Industries | 030311B |
| PBS (−) | Hyclone | SH30028.02 |
| Tryple Select | Gibco | 12563029 |
| Via-1 Cassette | Chemometec | 9410011 |

TABLE 21
Equipment.

| Equipment | CCN ID# |
|---|---|
| Accutrend Plus metabolite tester | 600-AP-002 |
| Clipster | 372-CL-002 |
| Eppendorf's BioFlo 320 bioprocess system | 530-BR-001 |
| NC-200 | 135-NC-005 |
| Sartorius Biowelder TC | 540-BW-001 |
| Sartorius pH meter PB-11 | 345-PH-001 |

### Experimental Procedure:

Harvesting of differentiated RPE cells, grown on T175 flasks, at the end of P1. Harvesting of differentiated RPE cells in the FFMD process, grown on recombinant human gelatin-coated T175 flasks (except in MCS 9, cells were thawed from the ICB and expanded on flasks prior to SUB inoculation). Inoculating the SUB with harvested RPE cells from an on-going RPE FFMD differentiation procedure in flasks, (except in MCS9 which originated from ICB), for one RPE expansion passage to P2 (with the exception of MCS11A inoculated at P1). Cells' origin for each run is described in Table 22, below.

TABLE 22
Cell origin in development studies

| | | # of passage | Passage |
|---|---|---|---|
| Study | Cell Origin | days in flasks | # in SUB |
| MCS 9 ¹ | HESPRO 04 GRPG LM521 P1 CS5 | 13 | 2 |
| MCS 11 A | FF run 05 GRPA P0 | 15 | 1 |
| MCS 11 B | FF run 05 GRPA P1 | 13 | 2 |
| MCS 13 | FF run 07 P1 | 14 | 2 |
| MCS 14 | FF run 08 P1 | 14 | 2 |

¹ Thawed cells from ICB.

SUB Inoculation. Selection of MC type, required cell number for inoculation and inoculation parameters (medium volume and agitation speed), were developed using Corning spinner flasks. Inoculation of the SUB with the harvested RPE cells on MC was optimized to 100,000-120,000 live cells/cm² of MC depending on the final yield of RPE cells obtained from harvesting the flasks. Inoculation medium (20% HS/DMEM) and MC are equilibrated for at least 20 minutes in the SUB at 37° C. prior to inoculation to allow the coating of the MC with HS to improve cell adherence, and achieve a homogenous MC suspension in the SUB. Other parameters, such as DO, pH, gassing and the medium replacement/additions, are as established during MCS1 to MCS8 studies.

### Agitation Speed

As the spinner flasks and SUB differ greatly in impellers' and vessels' sizes and geometry, agitation speed was established by applying the ‘constant impeller tip speed’ principle for maintaining a relatively constant shear force level during the transition from spinner flasks to SUB, while allowing sufficient mixing and oxygen transfer. ‘Constant tip speed’ was calculated as per the following formula Formula I:

$$Tip speed = π \times d \times N ,$$

where d=impeller's outer diameter (m)
   N agitation speed (rpm)

TABLE 23
SUB inoculation conditions

| # | Loop | SF | SUB |
|---|---|---|---|
| 1 | Agitation | 40 rpm | 22 rpm |
| 2 | Temp. | 37° C. | 37° C. |
| 3 | DO | NA | 30% |
| 4 | pH | NA | 7.25 |

Inoculation conditions were maintained for 4 days before medium replacement.

TABLE 24
SUB inoculation size per study

| | Total | Net live cell | Inoculation cell |
|---|---|---|---|
| | Inoculation | No. for SUB | density |
| Study | live cell No. | inoculation ¹ | (cells/cm²) |
| MCS 9 | 9.17 × 10⁸ | 8.86 × 10⁸ | 1.20 × 10⁵ |
| MCS 11 A | 1.26 × 10⁹ | 1.22 × 10⁹ | 1.13 × 10⁵ |
| MCS 11 B | 1.26 × 10⁹ | 1.22 × 10⁹ | 1.17 × 10⁵ |
| MCS 13 | 1.19 × 10⁹ | 1.15 × 10⁹ | 1.10 × 10⁵ |
| MCS 14 | 1.30 × 10⁹ | 1.26 × 10⁹ | 1.20 × 10⁵ |

¹ Approx. 3.3% of the total inoculation live cell no. was sampled from the SUB for various study controls as recorded in the protocols.

Expansion of RPE cells on MC in 3 L SUB

On day 4 post-inoculation the inoculation medium is replaced volume-per-volume (1.5L) with NUT(−)/HSA and cells' adherence to the MC was observed to validate cell attachment. Expansion of RPE cells was carried out under the fed-batch feeding regime and controlled parameters of temp., DO, pH, agitation and gassing. While the feeding regime, temp., and agitation speed have been established previously using T-flasks and SF, the environmental parameters—DO, pH and gassing—could not be scaled-up based on previous experience with the SF. Those parameters set points were developed and established, based on previous MCS1-8 runs and common practice.

As described previously, agitation speed scale-up was established by applying the ‘Constant impeller tip-speed’ principal, concurrent with our observation of the MC behavior, aiming to keep the MC suspended in the medium with minimal to no sedimentation. Agitation speed is first increased from 22 rpm to 29 rpm on Day 4, immediately following medium replacement with NUT(−)/HSA. This increase improves both temperature stability in the SUB and MC suspension which adds up to an overall better homogeneity of the culture yet keeps shear stress relatively low. Agitation is further increased to 40 rpm on day 7 and remains unchanged for the duration of the process, to improve MC suspension in the larger volume.

TABLE 25
Expansion phase SP

| # | Parameter | SF SP | SUB SP |
|---|---|---|---|
| 1 | Agitation | 50 rpm | 29 rpm (d4), |
| | | | 40 rpm (d7) |
| 2 | Temp. | 37° C. | 37° C. |
| 3 | DO | NA | 30% |
| 4 | pH | NA | 7.25 |

pH set-point was set to 7.25 with a 1.25 dead band, with no active control. pH gradually decreases over time during the process, from a relatively high pH of approx. 7.9 in the inoculation step (mostly contributed to the presence of HS), to approx. pH 7.3 at the end of expansion. pH was verified by using an external analog pH meter to measure the SUB's sample, and in case the actual pH value deviated in more than +0.05 pH units from the BioFlo 320 pH value, pH was re-standardized to the actual measured value. Feeding regime during RPE cell expansion was previously established as fed-batch, together with glucose addition. The culture is supplemented with 0.5L NUT(−)/HSA on 3 separate days, in intervals of 2-3 days, up to a final volume of 3 L. Glucose is supplemented to 2-2.5 gr/L (with 45% w/v d-glucose solution) based on the actual daily culture's glucose level as measured using the metabolite tester. Table 26 below details the schedule of medium replacement, fed-batch regime and harvesting of each run.

TABLE 26
Medium replacement, fed batch regime and harvesting schedule

| | Medium | 1^{st} Fed | 2^{nd} Fed | 3^{rd} Fed | |
|---|---|---|---|---|---|
| MCS # | replacement | Batch | Batch | Batch | Harvesting |
| MCS 9 | D4 | D6 | D10 | D12 | D13 |
| MCS | D4 | D7 | D10 | D11 | D13 |
| 11A | | | | | |
| MCS | D4 | D7 | D10 | D12 | D13 |
| 11B | | | | | |
| MCS | D4 | D6 | D8 | D11 | D14 |
| 13 | | | | | |
| MCS | D3 | D6 | D8 | D10 | D14 |
| 14 | | | | | |

D = Day

The optimal harvesting day was set to Day 14, therefore, a minimal expansion of 13-14 days has been determined for operation flexibility.

### Gassing Regime

Gassing regime via both the Overlay and Sparger is based on a mixture of 3 gasses—air, oxygen and carbon dioxide—which are actively controlled by the BioFlo system's automated 3-gas mix algorithm. The operating range was set based on common industry practice with hESC. The SUB has two aeration inlets. The main one is a pinhole sinter via which the 3-gas mixture, required for maintaining oxygen concentration (DO), is supplied. The second one is an overlay inlet for maintaining headspace aeration. Both inlets have pre-installed 0.2 μm 5 cm disc filters for filtration of incoming gasses. Small pore size, as in a sinter, produces small bubbles with high surface area, thus improving the gas transfer rate into the medium. However, such a high level of small bubbles may lead to the formation of foam which might lead to decreased gas exchange at the liquid-headspace border and eventually even clog the exhaust filter. The gas influx is very low and no formation of a significant foam layer has been observed during these studies.

TABLE 27
3-gas mix SP

| # | Loop | SP (SLPM) |
|---|---|---|
| 1 | Sparger | 0.002-0.050 |
| 2 | Overlay | 0.0-1.0 |

Vessel pressure affects the dissolved gases in the culture, thus affecting pH and DO. A decrease in overall gas pressure may cause a decrease in gas solubility and consequently lead to an increase in overall gas demand. The SUB is designed to operate under positive pressure and according to manufacturer recommendations, gas pressure in the SUB should not exceed 0.44 barg (6 psig). However, a positive flow of gases is necessary to maintain the culture, but as a relatively low gas flux is required during culturing of RPE cells (see Table 18 at lines 20 and 21, and Table 27) no critical pressure rise has been expected nor observed during these studies. Moreover, the SUB's pre-installed vent filter is designed to withstand a maximum pressure of up to 5 bar.

### Inoculation, Medium Replacement and Fed Batch Medium Additions

Maintaining the SUB as a closed-system, as opposed to RPEs expansion in flasks, require that all medium additions and replacements throughout the process will be carried out without exposing the culture to the external environment and will keep the culture in aseptic conditions. An automated Biowelder is used to weld medium-containing bags to the SUB's ports in a sterile and secure way. The single-use bags are pre-filled with the required medium in a biological safety cabinet before welded to the SUB.

### Harvesting

Filtration of RPE cells harvested from T-flasks and SF was established using a 40 μm grid mesh. Scaling up the procedure and establishing it as a ‘closed-system’ required using filter sizes of 50 μm or 60 μm, both with a surface area of 0.15 m². These filters are the nearest in pore sizes to the 40 μm grid mesh and thus assure that no particle larger than 60 μm will be present in the DS suspension.

Each SUB was filtered using 50 μm (SARTOPURE®) or 60 μm (VANGUARD®) high capacity filters that maintain a closed-system, for separating the RPE cells from the MC and to clear the solution from residual large cell aggregates and ECM, and quenched immediately after enzymatic incubation with TrypLE Select. Additionally, The SUB was sampled at the end of enzymatic incubation, immediately prior to quenching, and the sample was quenched and filtered using a 40 μm mesh grid, for assessing the scaled-up filtration process and obtaining a yield estimation of the SUB.

Before filtering the suspension, the filter is primed with approx. 0.5L of the quench solution and the incubated suspension is then passed through the filter via a peristaltic pump. The cell suspension is filtered intermittently, with the fraction of the cell suspension containing the largest visible aggregates passed through the filter during the final stage of filtration to minimize filter clogging and potential cell loss. This part of the filtration procedure was developed and established following the relatively low cell yield obtained in MCS9 (see Table 28, at Estimated versus final yields of MCS9-14).

### Results

The following results represent the scale up development studies of MCS 9, MCS 11 A, MCS11 B, MCS 13 and MCS 14. Results were obtained from the following assays: % Viability, % Recovery, Purity, HES Residual, biological activity (Potency), and Karyology.

Acceptance Criteria of QC assays

% Viability ≥70%. % Recovery: 100%±25% Live cells/ml, calculated relative to the targeted final batch concentrations of 2×106 cells/ml. Potency: Net TEER (day 14): >100 Ω*cm2; Basal VEGF/Apical VEGF Ratio (day 14): >1.00; Apical PEDF/Basal PEDF Ratio (day 14): >1.00. Purity: OpRegen® cells at P2 are double-positive for CRALBP and PMEL17≥95.00%. HES Residual: cells are double-positive for TRA1-60 and Oct-4<0.01000%. Karyology: less than three identical deletions or two identical additions to the chromosome.

### DO Trend

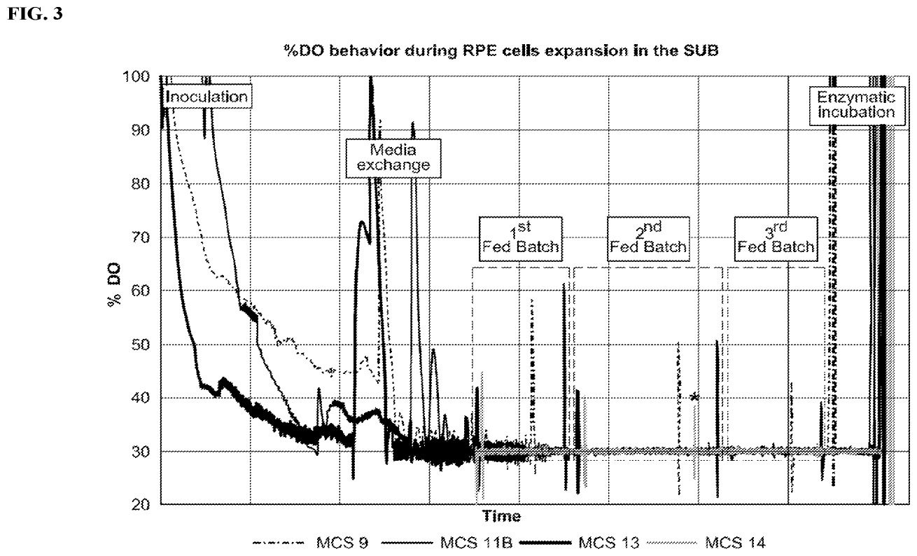

As shown in FIG. 3, a steep decline in % DO is apparent immediately following inoculation until medium is replaced. This % DO decline is indicative of a successful inoculation and viability. MCS 9 shows higher % DO values during inoculation due to its smaller inoculation size. In the 2^{nd} and 3^{rd} fed-batch, the relative fraction of added medium is smaller which results in diminished % DO spikes diminishing, since relatively less air enters the SUB with each fed-batch.

### Final DS Yields

TABLE 28
Estimated versus actual final yields

| | | | Harvesting | | | |
|---|---|---|---|---|---|---|
| | Inoculation | | | Estimated | | |
| | Total | Net live cell | Estimated cell | cell yield by | Final live cell | Final yield |
| | Inoculation | No. for SUB | No. by sample | sample | No. (DS post | (DS post |
| Study | live cell No. | inoculation ¹ | harvesting | harvesting | filtration) | filtration) |
| MCS 9 | 9.17 × 10⁸ | 8.86 × 10⁸ | 4.23 × 10⁹ | 4.77 | 2.37 × 10⁹ | 2.67 |
| MCS 11 A | 1.26 × 10⁹ | 1.22 × 10⁹ | 3.84 × 10⁹ | 3.15 | 4.02 × 10⁹ | 3.30 |
| MCS 11 B | 1.26 × 10⁹ | 1.22 × 10⁹ | 4.53 × 10⁹ | 3.71 | 3.91 × 10⁹ | 3.20 |
| MCS 13 | 1.19 × 10⁹ | 1.15 × 10⁹ | 5.40 × 10⁹ | 4.70 | 4.20 × 10⁹ | 3.65 |
| MCS 14 | 1.30 × 10⁹ | 1.26 × 10⁹ | 5.08 × 10⁹ | 4.03 | 2.03 × 10⁹ | 1.61 |

¹ Approx. 3.3% of the total inoculation live cell no. was sampled from the SUB for various study controls.

Each SUB was sampled (60 mL) at the end of enzymatic incubation, immediately prior to quenching, and the sample was quenched and filtered using a 40 μm mesh grid, for assessing the scaled-up filtration process and obtaining a yield estimation of the SUB. The average estimated yield of all studies (presented in Table 28) was approx. 4.09, close to their actual final yields measured in the DS post-filtration, with an average final yield of approx. 2.87, which constitute approx. 30% average loss-on-filter. MCA11 A, MCS11B and MCS13 achieved final average DS yields of approx. 3.35 post filtration, which resembles their respective estimated yields. The final yield of study MCS 9, which was obtained prior to establishing the requirement to prime the filter before filtration, was approx. 44% lower than its estimated yield (4.78 vs. 2.68). MCS 14, which was subjected to a different fed-batch regime than the other studies, had a final yield that was 60% lower than expected.

### % Viability and % Recovery Assessments

5 vials of each study (2×10⁶ cells/ml) were thawed and assessed for % Viability and % Recovery. Results are summarized in Table 29.

TABLE 29
% Viability and % Recovery results

| | | | Avg. % | Avg. % | | | | |
|---|---|---|---|---|---|---|---|---|
| | | | Viability | Recovery | Avg. study | SD % | Avg. study | SD % |
| Cell/ml | Study ID | Vial # | per vial | per vial | % Viability | Viability | % Recovery | Recovery |
| 2 × 10⁶ | MCS 9 | 1 | 91 | 85 | 90 | 1.1 | 88 | 6.0 |
| | | 2 | 92 | 89 | | | | |
| | | 3 | 90 | 97 | | | | |
| | | 4 | 89 | 81 | | | | |
| | | 5 | 90 | 90 | | | | |
| | MCS 11 A | 1 | 95 | 90 | 95 | 1.1 | 89 | 3.1 |
| | | 2 | 97 | 85 | | | | |
| | | 3 | 94 | 92 | | | | |
| | | 4 | 95 | 86 | | | | |
| | | 5 | 96 | 91 | | | | |
| | MCS 11 B | 1 | 95 | 82 | 96 | 0.9 | 78 | 4.0 |
| | | 2 | 97 | 73 | | | | |
| | | 3 | 96 | 76 | | | | |
| | | 4 | 95 | 82 | | | | |
| | | 5 | 95 | 76 | | | | |
| | MCS 13 | 1 | 95 | 111 | 95 | 1.9 | 104 | 8.9 |
| | | 2 | 94 | 96 | | | | |
| | | 3 | 97 | 107 | | | | |
| | MCS 14 | 1 | 96 | 109 | 95 | 0.9 | 105 | 5.7 |
| | | 2 | 96 | 105 | | | | |
| | | 3 | 95 | 102 | | | | |

% Viability and % Recovery of all vials from all studies immediately post-thawing met OPREGEN® TAI acceptance criteria (presented in Table 29). % Viability was maintained at NLT 90%. % Recovery was NLT 78% with an average of 90%.

### Biological Activity (Potency) Assessment

Thawed cells were assayed for biological activity and results are summarized in Table 30.

TABLE 30
Potency assay results

| | AVE Net TEER | | PEDF | | VEGF | |
|---|---|---|---|---|---|---|
| | (Ω*cm²) | | Apical/Basal | | Basal/Apical Ratio | |
| | Day 14 | | Ratio (day 14) | | (day 14) | |
| | Current | Direct | Current | Direct | Current | Direct |
| Sample | Method | Method | Method | Method | Method | Method |
| MCS 9 | 673 | 198 | 4.39 | 4.26 | 2.30 | 1.85 |
| MCS 11 A | 245 | 323 | 4.40 | 5.52 | 1.34 | 1.88 |
| MCS 11 B | 293 | 262 | 4.00 | 4.36 | 1.89 | 1.77 |
| MCS 13 | 275 | 652 | 11.46 | 7.18 | 1.84 | 2.49 |
| MCS 14 | 298 | 428 | 5.61 | 6.54 | 2.19 | 2.20 |

All studies have met the biological activity assay acceptance criteria in both potency methods.

### Purity Assay Results

Thawed cells were assayed for purity and results are summarized in the Table 31 below.

TABLE 31
Purity (CRALBP/PMEL17) assay results

| | % CRALBP |
|---|---|
| Batch | PMEL17 |
| MCS 9 | 86.37 |
| MCS 11 A | 97.73 |
| MCS 11 B | 96.65 |
| MCS 13 | 96.07 |
| MCS 14 | 90.58 |

RPE cells obtained from studies MCS 11A, MCS 11B and MCS 13 met the purity assay's acceptance criteria while cells from MCS 9 and MCS 14 have failed. Note that both MCS 9's and MCS 14's fed batch regime, were different from the other studies. Moreover, both MCS 9 and MCS 14 gave the lowest final yields which might have affected the final cell population.

% hESC Residual Assay Results

Thawed cells were assayed for % hESC residuals and results are summarized in Table 32.

TABLE 32
% hESC Residual (Oct-4/TRA1-60) assay results

| Batch | % Oct- | Acceptance |
|---|---|---|
| | 4/TRA1-60 | (<0.01000%) |
| MCS 9 | 0.00052 | Passed |
| MCS 11 A | 0.00004 | Passed |
| MCS 11 B | 0.00036 | Passed |
| MCS 13 | 0.00157 | Passed |
| MCS 14 | 0.00433 | Passed |

RPE cells obtained from all five studies met the % hESC Residual assay's acceptance criteria (Table 32).

### Karyotyping

Frozen vials at P2 were thawed, cultured for two passages and fixed for karyotype analysis. Karyotype of cells from each of these studies was normal; No 3 identical chromosomal depletions or 2 identical additions were found. One exception was with MCS11A in which isoq20 was found and this result is under investigation.

### Discussion

To establish a scaled up process of OPREGEN® production, RPE cells were cultured on MC for one expansion passage, in a 3 L SUB, under controlled and monitored conditions. Five consecutive development studies—MCS 9/11A/11B/13/14—were executed under the suggested scaled-up process parameters, which were gradually improved during earlier scale-up studies MCS1-8. These parameters are shown to deliver a robust and reproducible scaled-up process. Examination of the DO trends in these studies reveal an almost identical behavior; a steep decline immediately following inoculation, which continues for the first couple of days is indicative of a successful inoculation and % viability. % DO then stabilizes at SP 30%, followed by intermittent spikes that correspond with medium replacement and subsequent fed batch medium additions. The fed-batch regimen has been shown to produce better final cell yields, when fed in 2-3 days intervals (as presented in Table 26—at medium replacement, fed batch regime and harvesting schedule); a 4-day interval (MCS 9 & MCS 14) might induce some stress on the culture which subsequently affects filtration efficiency and final population composition with regards to RPE maturation.

During development, priming the filter with the quenching solution, prior to MC-cell separation in order to minimize cell loss, was shown to be an improvement. Furthermore, the improved filtration procedure, which includes passing the large aggregates-containing suspension fraction only in the final stages of the filtration, rather than continuously mixing and filtering the entire bulk volume (as executed in MCS 9), has proven vital for obtaining higher cell yields. As for the significantly lower final yield obtained in MCS 14, it might be due to the different feeding regimen, during that run the 2^{nd} fed batch was supplemented 4 days following the 1^{st}, instead of 3 days. This might have contributed to larger ECM production by the RPE cells, which finally trapped more matured cells during harvesting and filtration.

The developed scaled-up process was shown to be effective and reproducible with final average cell yields of approx. 3.35 (See Table 28, where the average number of 3.35 relates to MCS11A_11B and 13), given that the cells are expanded and obtained under the suggested fed batch regimen, (no more than 3 days between feeding), and the improved filtration procedure (i.e., MCS 11A, MCS 11B and MCS 13).

Purity assay results (As presented in Table 31) of studies MCS 11A, MCS 11B and MCS 13, met assay's acceptance criteria, while MCS 9 and MCS 14 failed to meet these acceptance criteria. The reason is due to different parameters in MCS 9's and MCS 14's processes. Their respective actual FACS diagrams show a broader population of cells exhibiting lower PMEL values, which are typical of less mature (younger) RPE cells. These relatively broader-then-usual (i.e., compared to MCS 13) populations of less mature cells, most likely stem from a non-optimized harvesting procedure. As more mature cells produce more ECM, this makes the mature RPE cells harder to detach from the MC during enzymatic incubation with TrypLe Select compared to less mature RPE cells. Consequently, these matured cells are trapped within the ECM aggregates during filtration; and hence the DS becomes relatively enriched with less mature RPE cells post-filtration.

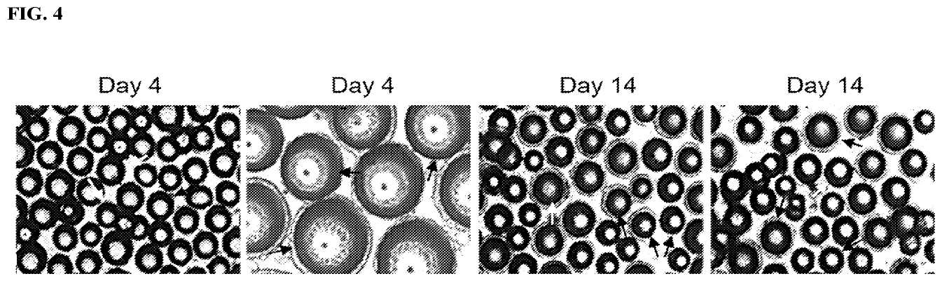

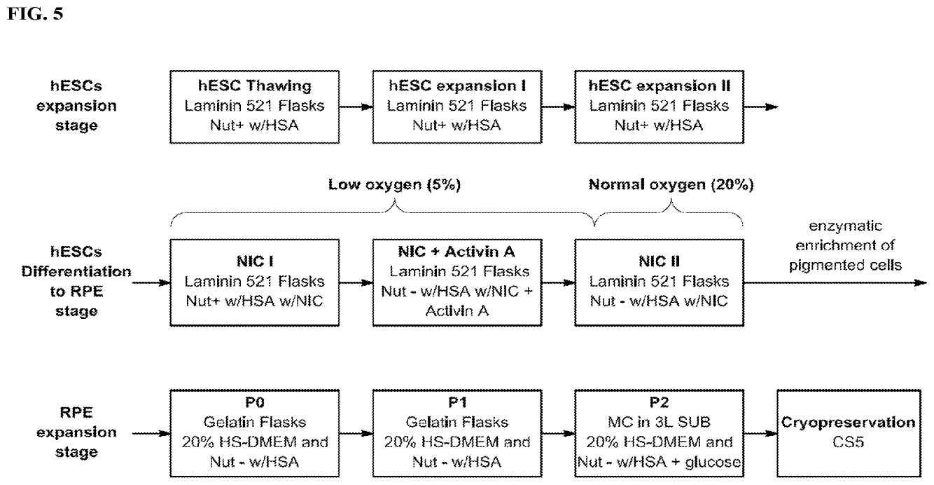

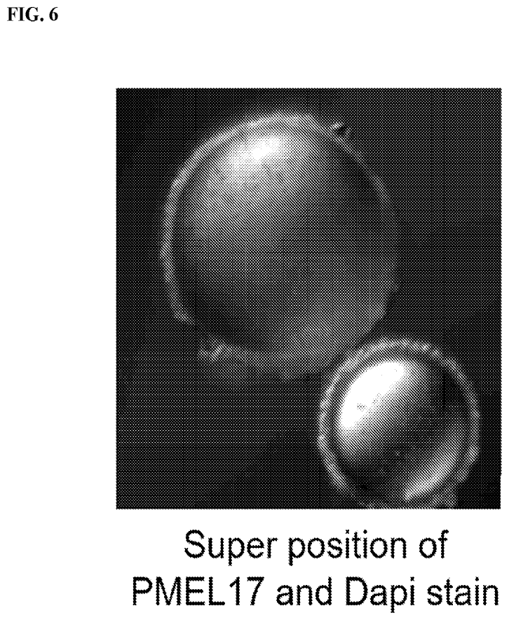

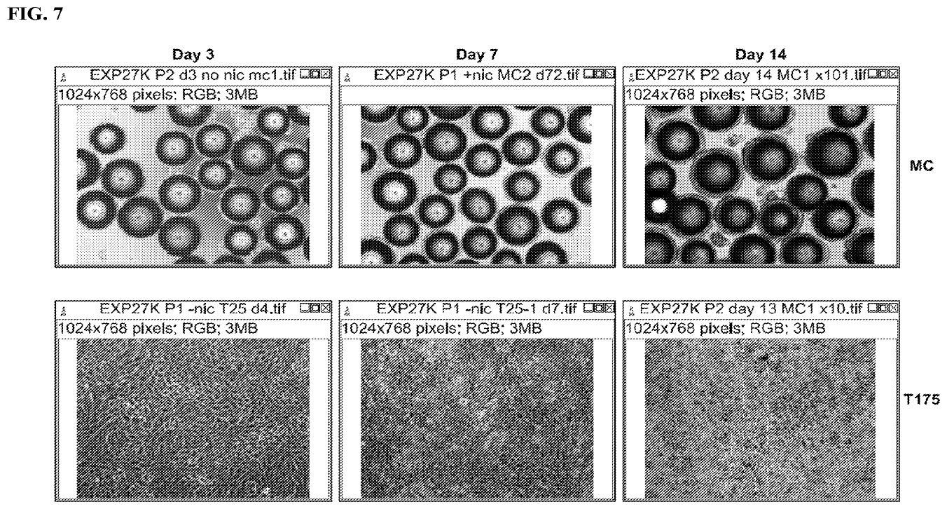

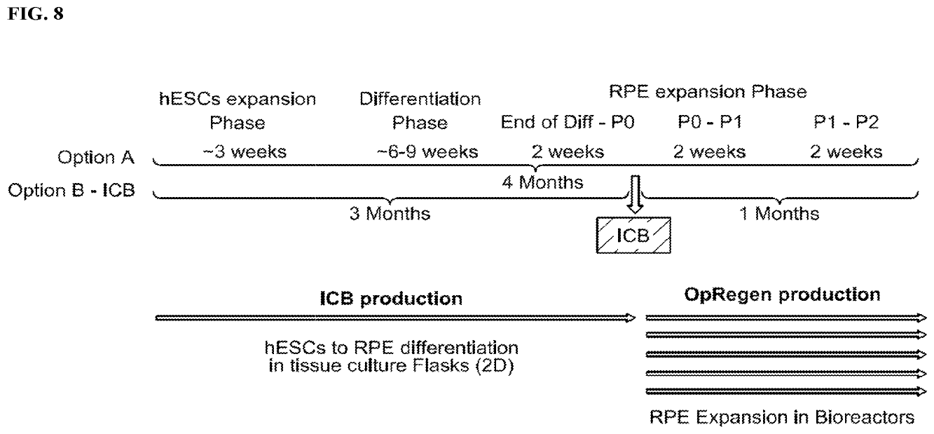

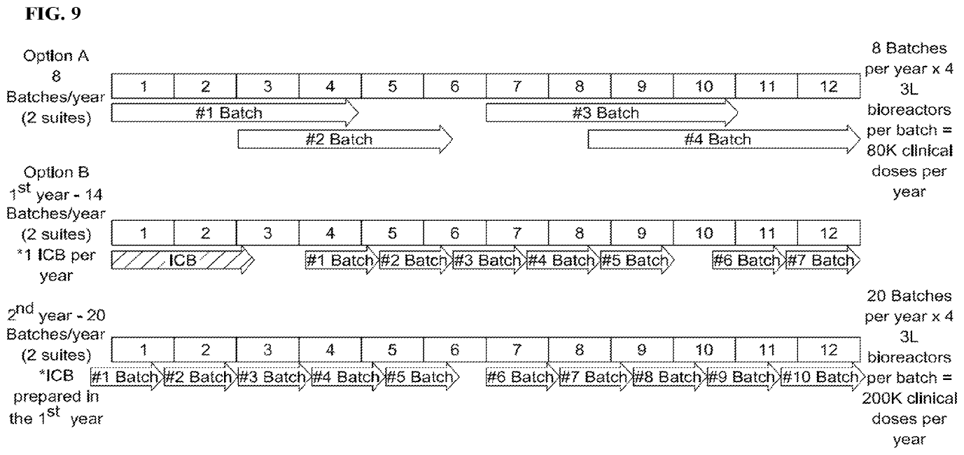

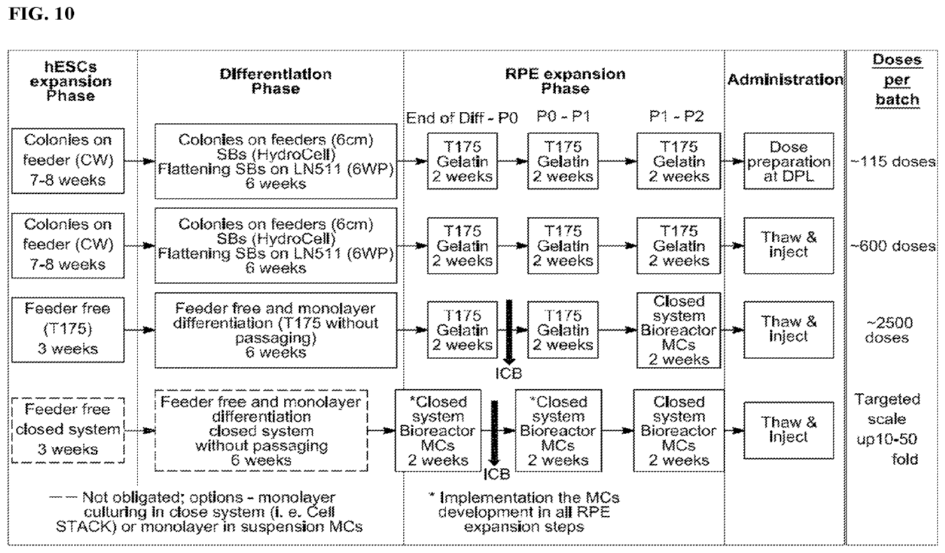

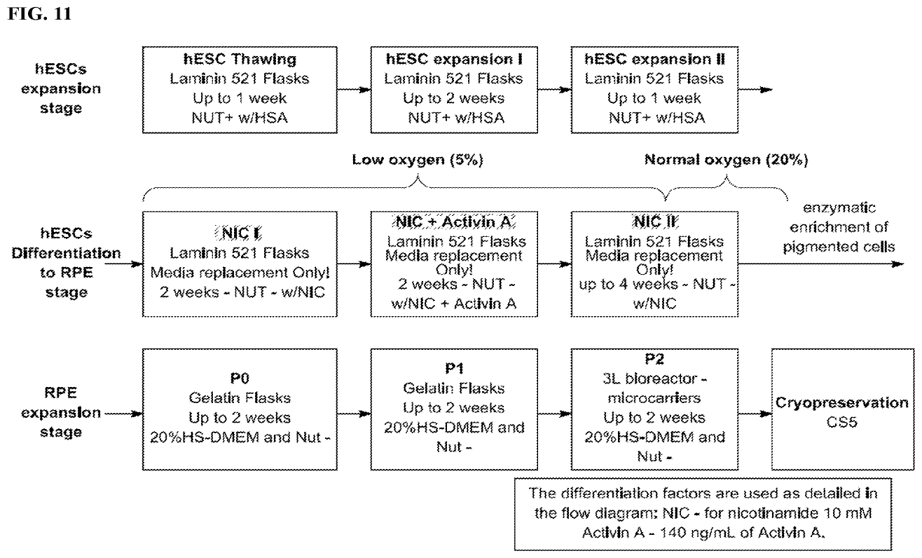

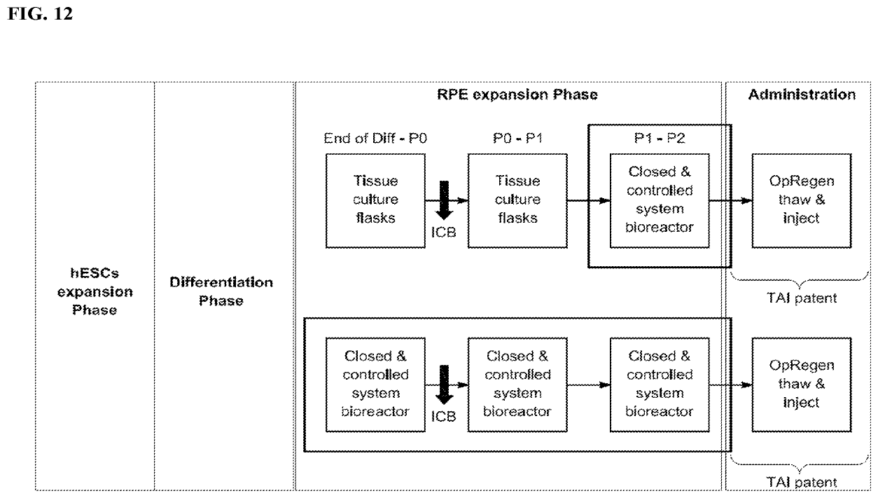

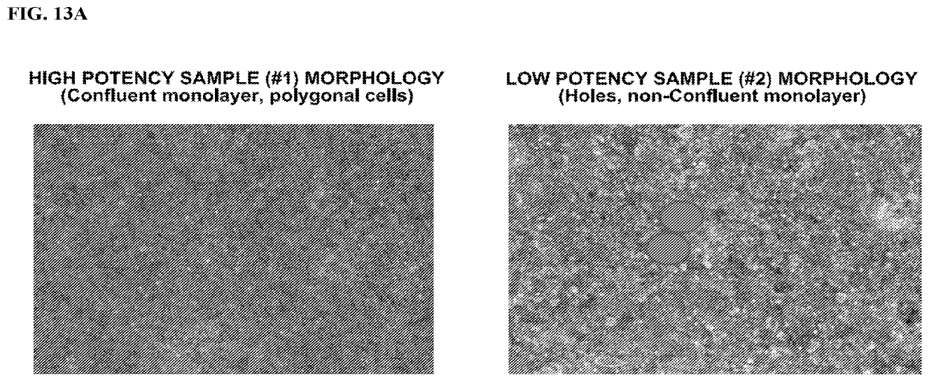

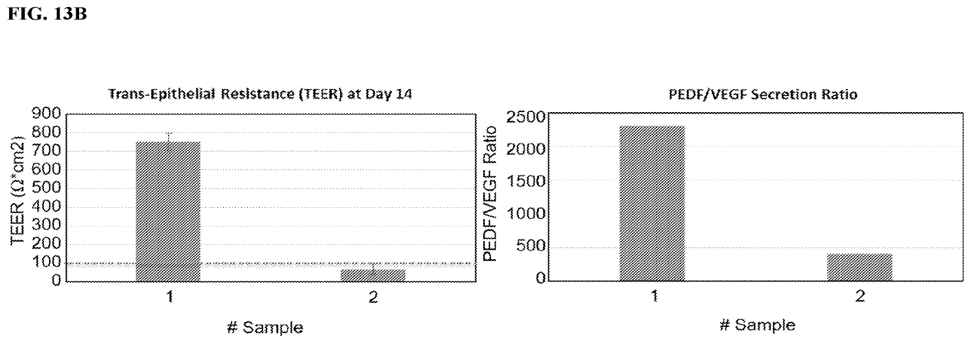

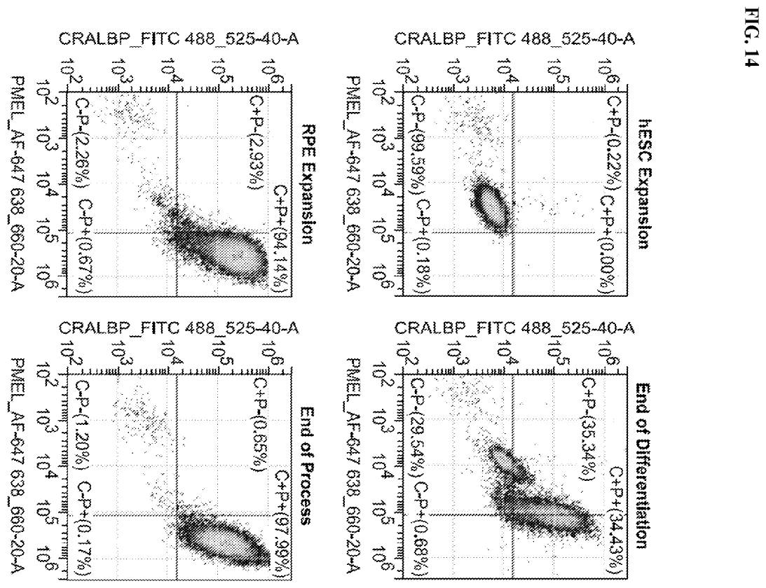

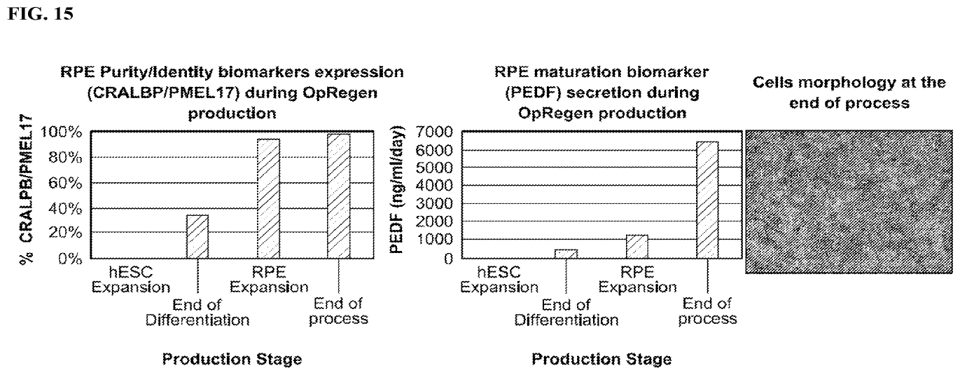

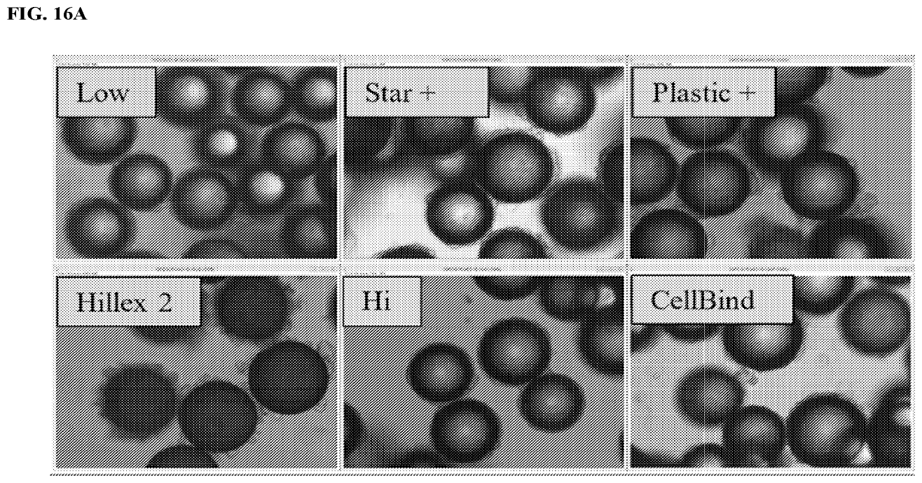

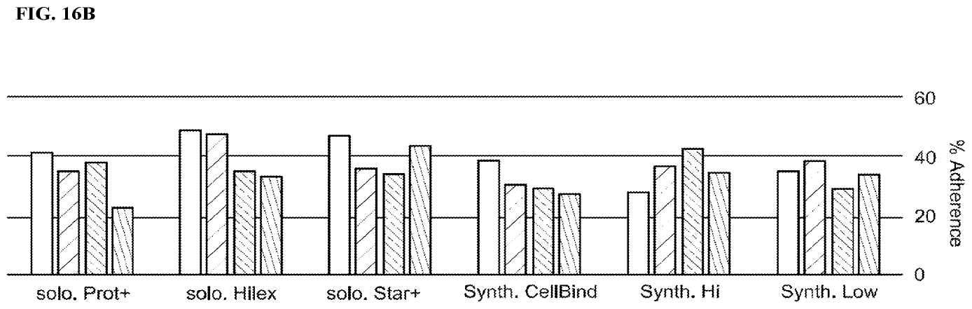

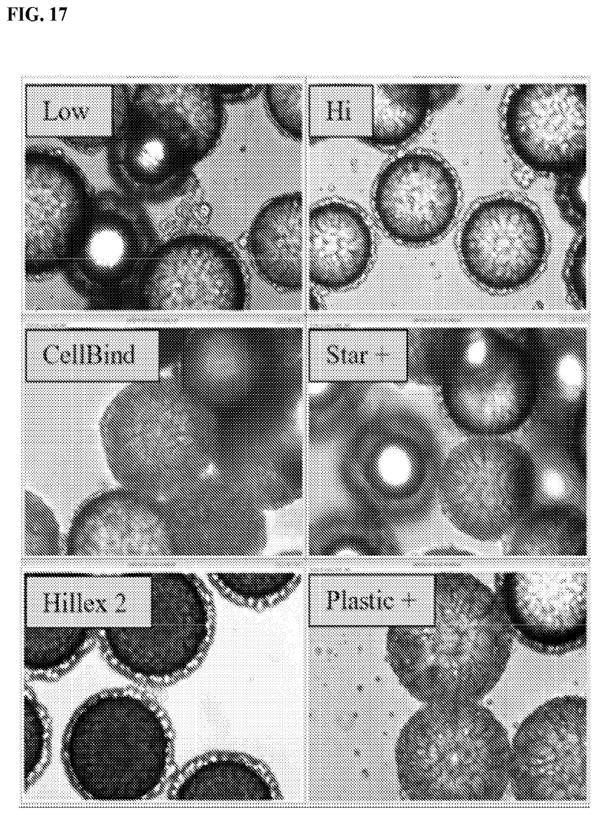

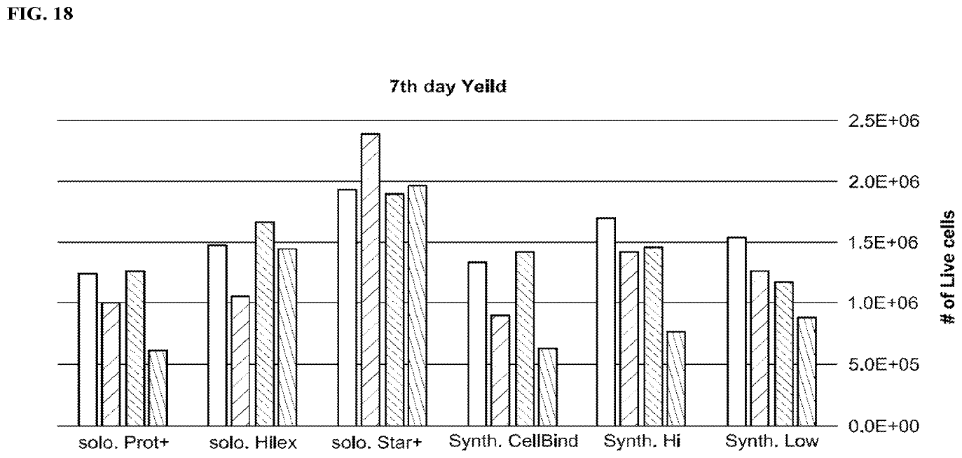

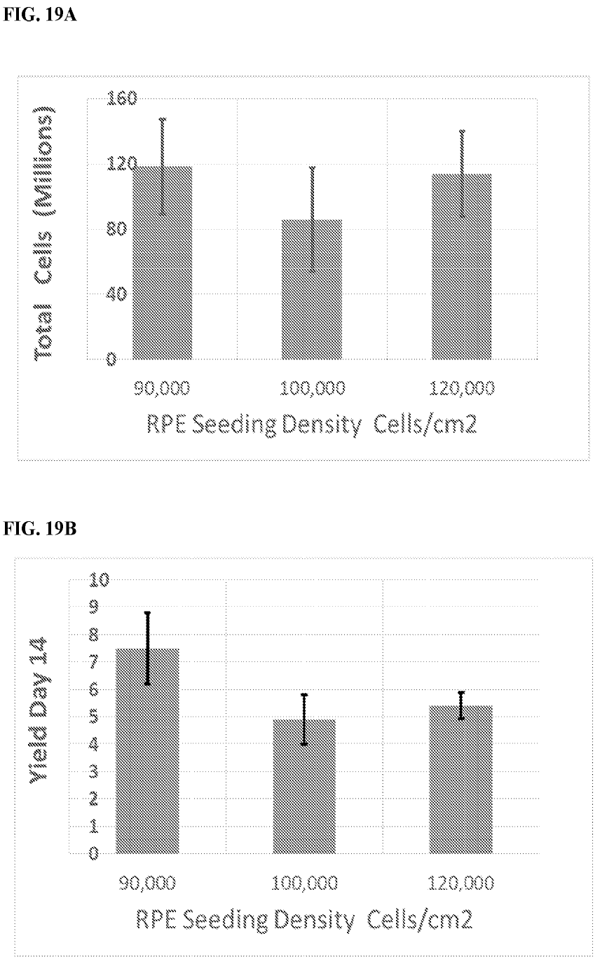

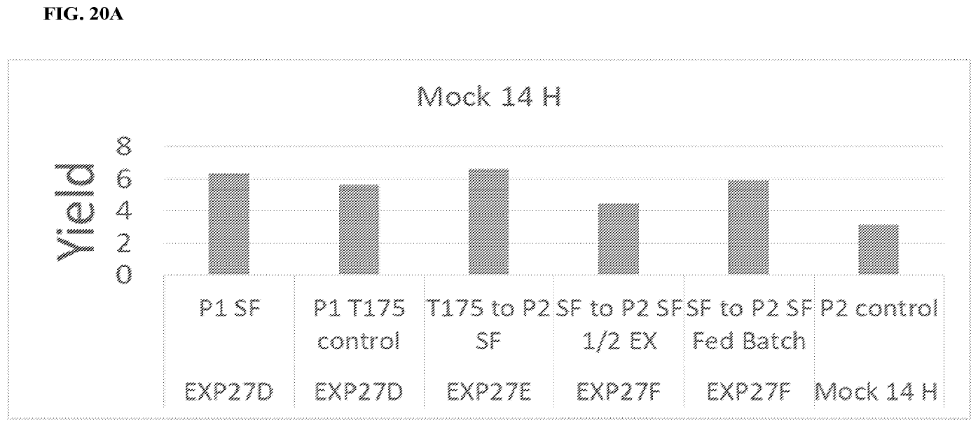

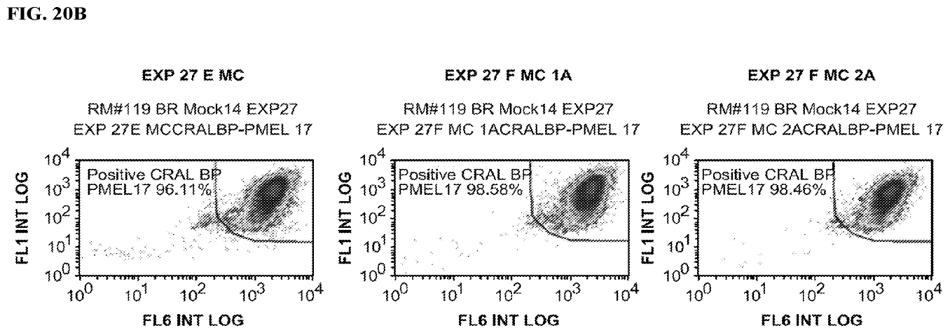

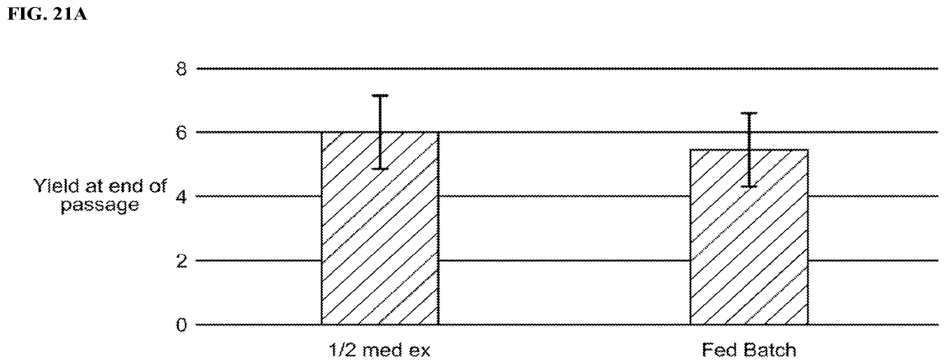

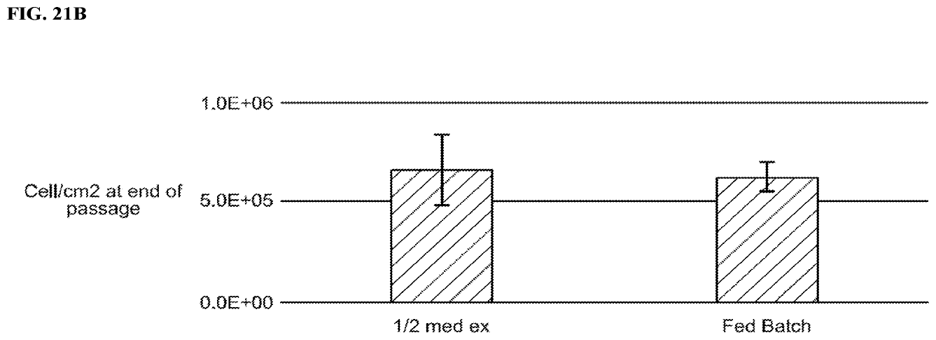

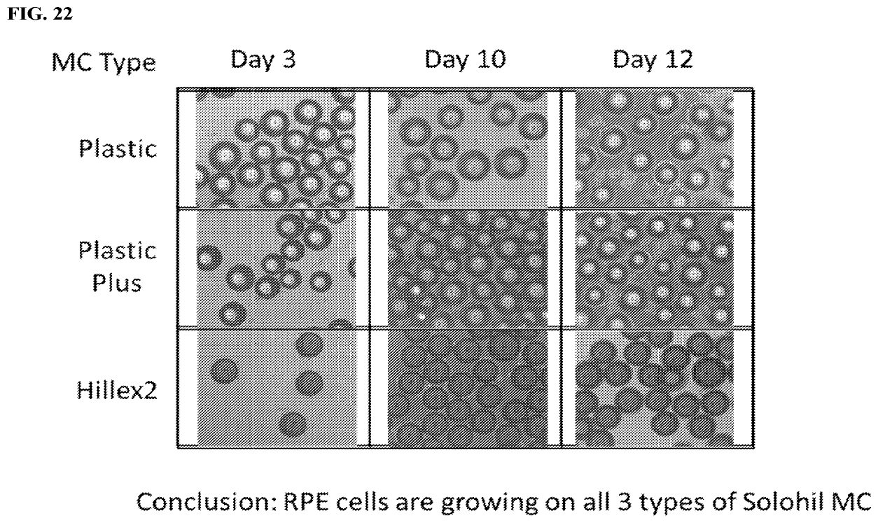

Studies MCS 11A, MCS 11B and MCS 13 met the purity assay's acceptance criteria, showing that the optimized fed batch regimen and filtration procedures are required for successful scaled up RPE expansion. Maturation variations in the final RPE population are likely to result in inherent heterogeneous MC cell densities, which is probably a consequence of the inoculation. Being a 3D stirred cell expansion platform, the MC are prone to uneven distribution of cells during inoculation as opposed to 2D static tissue-culture flasks, (as shown in FIG. 4). The versatile maturity of the final population will likely be beneficial to the RPE cells.

% hESC Residual assay results met OpRegen® TAI acceptance criteria. All studies have met the Biological Activity (Potency) assays' acceptance criteria under both assays' methods. Karyotype analyses results of four (4) out of the five (5) studies were normal; One of the studies showed an abnormal karyotype (Isoq20 (3/50)) and this was investigated.

To summarize, the bioprocess system in general and the scaled-up expansion platform in particular, are able to maintain and support a robust RPE cell expansion and reproducible process, by maintaining the required OPREGEN® quality attributes and characteristics.

### Conclusions

The BioBlu 3C SUB combined with the BioFlo 320 bioprocess system has demonstrated robustness in maintaining and supporting the expansion of RPE cells on MC in the current OPREGEN® process. Approximately 4×10⁹ OPREGEN® TAI cells (after final filtration) can be obtained from culturing RPE cells on MC with a surface area of 10.8×10³ cm², in a 3 L SUB, under controlled and monitored conditions. The scaled-up filtration procedure has been shown to be effective and reproducible when the established cell expansion process' fed batch regimen and the improved filtration procedure were implemented, resulting in the final DS cell yield of approx. 3.35.

TABLE 33
Solution volumes, MC amount and process parameters setpoints

| | | |
|---|---|---|
| # | Parameter | Value/SP |

Cells per Inoculation

| | | |
|---|---|---|
| 1 | Target cell density (cells/cm²) | 50K-200K |
| 2 | Total inoculation cell No. (×10⁶) for a final conc. of 100,000- | 500-2,000 |
| | 120,000 cells/cm² | |
| 3 | Total inoculation cell suspension volume (mL) (5 × 10⁶ cells/mL) | 500-100 |

MC Details

| | | |
|---|---|---|
| 4 | Amount per BioBlu 3C (gr.) | 10-60 |
| 5 | MC concentration (cm²/ ml) | 2-10 |
| 6 | Total surface per SUB (cm²) | 8,000-15,000 |

Inoculation medium ingredients

| | | |
|---|---|---|
| 7 | Volume of MC in DMEM suspension per BioBlu 3C (mL) | 400-1000 |
| 8 | Complementary DMEM volume (mL) | 100-500 |
| 9 | Human Serum (HS) (mL) | ~100-500 |
| | | (~6%-34%) |
| 10 | L-Glutamine (mL) | 5-30 (0.3-2%) |
| 11 | Pen-Strep (P/S) (mL) | 1-10 |
| 12 | Total inoculation volume (mL) | 500-5000 |

Fed Batch medium

| | | |
|---|---|---|
| 13 | Inoculation medium replacement solution NUT (-) with HSA (ml) | 500-5000 |
| 14 | Fed batch medium NUT (-) with HSA on Days 7, 10 and 12 (ml., | 100-1000 |
| | each) | |

Process Parameters

| | | |
|---|---|---|
| 15 | Agitation (rpm) | 10-30 (d0), |
| | | 10-40 (d4), |
| | | 20-50 (d7) |
| 16 | Temp. | 37° C. |
| 17 | DO | ≥30% |
| 18 | pH | 7.25 ± 1.25 |
| 19 | Sparger (SLPM) | 0.002-0.050 |
| 20 | Overlay (SLPM) | 0.0-1.0 |

### Example 6

The purpose of this example is to summarize the OPREGEN® Non-GMP engineering run of a GMP FF seed lot bank.

The OPREGEN® manufacturing process towards commercial production includes development of a Feeder Free and Monolayer Differentiation (FFMD) process. The FFMD process should be highly controlled, with minimal aseptic risk, and should be robust and less dependent on spontaneous differentiation steps.

The developed OPREGEN® commercial process relies on four stages of cells: first stage—Feeder Free (FF) human Embryonic Stem Cells are expanded on Laminin521 (LN521) for 3 weeks; second stage—FFMD procedure of hESCs, which differentiate the hESCs into RPE cells and developed based on the current OPREGEN® Thaw and Inject (TAI) process, is performed for 6-7 weeks; third stage—RPE expansion for two passages (P0, P1), currently done on gelatin coated flasks for additional 4 weeks as was done for OPREGEN® TAI; fourth stage—large scale culturing of RPE cells on microcarriers (MC) in a semi-automated controlled closed-system (Eppendorf's BioFlo 320 console that monitors BioBlu Single-Use Bioreactor (SUB)).

### Abbreviations and Definitions

CoA—Certificate of Analysis
CS5—CryoStor 5
DMEM—Dulbecco Modified Eagle Medium
DO—Dissolved Oxygen
Fed-batch—culture technique in biotechnological processes where one or more nutrients are fed to the bioreactor during cultivation and in which the product remains in the bioreactor until the end of the run
FF—Feeder Free
FFB—Feeder Free Bank
FFMD—Feeder Free and Monolayer Differentiation
GRP—group
hESCs—Human Embryonic Stem Cells
HS—Human Serum
HSA—Human Serum Albumin
IPCs—In Process Controls
LN521—laminin521
MC—Micro Carriers
MCB—Master Cell Bank
NIC—nicotinamide
Nut (−) w/HSA—NutriStem minus containing HSA
Nut+w/HSA—NutriStem plus containing HSA
PDL—Population Doubling Level
POC—Proof of Concept
QC—Quality Control
R&D—Research and Development
RPE—Retinal Pigmented Epithelium
RPM—Revolutions Per Minute
SD—Standard Deviation
TAI—Thaw and Inject
TEER—Transepithelial electrical resistance
TS—TrypLE Select
V—version
w/—with
w/o—without

### Procedures and Methods:

Cells: Cryopreserved in CCN GMP facility.

### Experimental Procedure

hESCs expansion and hESCs thawing

One vial of frozen cells was thawed and seeded at 6,000 live cells/cm² on 5 μg/ml LN521 coated dishes, in Nut+w/HSA medium. hESCs were cultured. When cell culture reached >50% confluency, the cells were harvested, counted and seeded at 3,500 live cells/cm² for hESCs expansion I.

hESCs Expansion I

hESCs were passaged for one additional passage on 5 μg/ml LN521 coated dishes with Nut+w/HSA medium. When cell culture reached >50% confluency, the cells were harvested, counted and seeded at 4,000 live cells/cm² for hESCs expansion II. In addition, a sample from the harvested hESCs was evaluated for pluripotency markers.

hESCs Expansion II

hESCs were seeded on 5 μg/ml LN521 coated dishes with Nut+w/HSA medium. Cells morphology and confluence were evaluated, starting from day 6 of culturing until cell culture reached >80% confluence. In addition, one equivalent flask (cultured under the same conditions), was harvested for testing pluripotency markers.

hESC Differentiation to RPE

### NIC I

At the end of hESCs expansion II, OPREGEN® differentiation process was initiated by changing the medium from Nut+w/HSA to Nut−w/HSA, supplemented with 10 mM NIC and by culturing cells in 5% O₂, 5% CO₂ and at 37° C. for 2 weeks.

### NIC+Activin A

Cells were cultured for two weeks with Nut−w/HSA, supplemented with 10 mM NIC and 140 ng/ml Activin A in 5% O₂, 5% CO₂ and at 37° C. At the end of 14 days, samples of the medium were collected for factors secretion tests.

### NIC II

Cells were cultured for 5 days with Nut−w/HSA, supplemented with 10 mM NIC in 5% O₂, 5% CO₂ and at 37° C. until lightly pigmented areas were observed under binocular microscope. Then, cells were cultured for 10 more days in normoxia (20% O₂, 5% CO₂ and 37° C.). At the end of the 15 days (end of differentiation), morphology of differentiating cells was evaluated, samples of the medium were collected for factors secretion tests and cells were harvested using TS and seeded on 0.1% rh-gelatin coated dishes with 20% HS-DMEM at 60,000 live cells/cm², for P0 of RPE expansion stage. In addition, a sample of cells was tested for RPE purity/identity.

### RPE Expansion

### RPE in T175 Flasks (P0, P1)

Cells were cultured in 20% HS-DMEM for 4 days and in Nut−w/HSA medium for 11 additional days for P0 (until reaching 100% confluency and RPE polygonal typical morphology), in 5% CO₂ and at 37° C. (total of 15 days at P0). At the end of P0, cells were harvested, seeded on 0.1% rh-gelatin coated dishes at 120,000 live cells/cm². In addition, cells were evaluated for RPE purity/identity and residual hESCs. Cells were cultured for additional 13 days at P1 before harvesting and seeding for P2.

### RPE in SUB (P2)

Harvested P1 cells were inoculated in SUB at optimal conditions. 20% HS-DMEM seeding medium with MC, was equilibrated in the SUB for at least 20 minutes. Afterward, cells were inoculated at 1.26×10⁹ live cells per 10,800 cm² of MC (as per 117,000 live cells/cm² of MC), in inoculation conditions of 37° C.; 22 rpm agitation; 30% DO; pH 7.25. After 4 days, the 20% HS-DMEM was changed to Nut (−) w/HSA medium, while expansion conditions of the RPE cells remain the same, except the agitation speed (29 rpm at days 3-7 and 40 rpm at days 7-14). Feeding regime during the P2 expansion stage was performed as fed-batch, together with glucose addition. The cell culture is supplemented with 0.5 L Nut (−) w/HSA medium on 3 separate days (7, 10 and 12), up to a final volume of 3 L. The glucose is supplemented to 2.5 g/L (with 45% w/v d-glucose solution), based on the actual daily culture's glucose level, as measured using the metabolite tester. At the end of 13 days of culturing, SUB was harvested and RPE cells were filtered using the 60 μm filter (Vanguard) and cryopreserved at 2×10⁶ cells/vial.

TABLE 34
Batch release tests for FFMD large scale OPREGEN ®production process

| | | Acceptance |
|---|---|---|
| Test | TEST | Criteria |
| Viability | NC-200 Cell Counting | ≥70% |
| Total Cell | NC-200 Cell Counting | 1.5 × 10⁶-2.5 × 10⁶ |
| Number/Vial | | |
| hESCs residuals | FACS for the absence | <0.01% of double |
| | of hESC markers | positive cells |
| RPE | FACS for RPE Purity | ≥95% of double |
| purity/identity | | positive cells |
| Polarization | Net TEER | ≥100 Ω*cm² |
| | PEDF Apical to Basal | >1 |
| | Ratio | |
| | VEGF Basal to Apical | >1 |
| | Ratio | |
| Karyotype | Giemsa banding | Normal |
| Analysis | | |

### Results

### In Process Controls

Pluripotency. Expression of pluripotency markers (SSEA-5/TRA-1-60, Oct-4/Nanog) was tested at the end of each hESCs expansion stages, Percentage of double positive cells in each assay was measured by FACS and shown in Table 35, below.

TABLE 35
Expression of pluripotency markers
along hESCs expansion stage

| End of hESCs expansion I | | End of hESCs expansion II | |
|---|---|---|---|
| % SSEA-5/ | % Oct-4/ | % SSEA-5/ | % Oct-4/ |
| TRA-1-60 | Nanog | TRA-1-60 | Nanog |
| 97.91 | 98.09 | 95.36 | 96.96 |

Expression of pluripotency markers was high over all hESCs expansion stage—percentage of SSEA-5/TRA-1-60, double positive cells was over 97.91% and 95.36% and % of Oct-4/Nanog double positive cells was 98.09% and 96.96%. Results showed that the cultured hESCs maintained their pluripotency prior to FFMD procedure initiation. Mild reduction in expression of markers observed at the end of hESCs expansion step I versus the end of hESCs expansion step II is related to the intended higher seeding density and confluence of the cells, before initiation of FFMD, which is required for the differentiation process.

### Purity and Identity

RPE markers (CRALBP/PMEL17) expression was tested along different stages of FFMD and RPE expansion phases.

TABLE 36
Expression of RPE markers along RPE differentiation and PO.

| End of differentiation (end of NIC II) | End of PO |
|---|---|
| (% of double positive cells) | (% of double positive cells) |
| 45.96 | 97.96 |

% of RPE cells represented by % of CRALBP/PMEL17 positive cells. % of RPE cells was 45.96% at the end of differentiation stage. After passaging and enrichment of the RPE cells at the end of P0, % of RPE cells was 97.96% and met the batch release acceptance criteria at this point (>95.00% of RPE cells).

Residual hESCs

For determining residual hESCs, RPE cells were stained for hESCs pluripotency markers TRA-1-60/Oct-4 at the end of P0. % of TRA-1-60/Oct-4 was below limit of detection (<0.0004%).

### PEDF Secretion Along Process

In previous OPREGEN® TAI V1.1 runs an increase of magnitude in PEDF levels secreted to culture media was observed at the final differentiation stage (end of Activin A, end of differentiation) and at early RPE expansion stage (end of P0); thus those IPCs were selected to be tested for PEDF concentration in FFMD process and in this non-GMP engineering run—FFMD large scale OPREGEN® production process. Those 3 points for evaluation of PEDF found to be significant and indicative points as IPCs for FFMD large scale OPREGEN® production process. PEDF concentration in culture media was evaluated at three points according—end of NIC+Activin A, end of differentiation (end of NIC II) and end of P0. Results are presented in Table 37.

TABLE 37
Secretion of PEDF to culture media along process
PEDF Concentration (ng/ml/day)

| End of NIC & | End of differentiation | End |
|---|---|---|
| Activin A | (end of NIC II) | of P0 |
| 256.19 | 2,104.01 | 4,687.02 |

As expected, PEDF levels elevated as cell population gets purer and RPE population is more mature (along the progression of the process). Between the end of NIC & Activin A step and the end of differentiation step, PEDF levels were increased (over 10 times higher); Between end of differentiation and the end of P0, the elevation in PEDF was moderate (over 2 times higher).

### PDL Along Process

During RPE differentiation, yield value was calculated for determining how many RPE cells were harvested from each hESC seeded for differentiation process.

TABLE 38
PDL of cells during differentiation and RPE expansion
PDL

| End of differentiation | End of P0 | End of P1 | End of P2 | Total |
|---|---|---|---|---|
| 8 | 4 | 2 | 2 | 16 |

Number of cell doublings along process was ˜16, similar to former runs of FFMD protocol.

### Batch RELEASE

### % Viability and Total Cell Number/Vial

Frozen vials at the end of P2 (MCS 11B) at concentration of 2×10⁶ live cells/mL/vial were thawed and tested for % Recovery and % Viability.

TABLE 39
% Viability and % Recovery of thawed vials.

| Average | SD | Average | SD recovery | # of vials tested |
|---|---|---|---|---|
| viability (%) | viability (%) | recovery (%) | (%) | (n) |
| 96 | 0.9 | 78 | 4.0 | 5 |

Results have met the acceptance criteria of % Recovery over 75% and % Viability over 70%. The relatively low % Recovery values might be a consequence of the long DP incubation time (over two hours) due to cryopreservation of several groups in parallel. Moreover, counting of cells in CS5 just before vialing pointed a low concentration of live cells (1.75×10⁶ live cells/mL). Normalization of the cell counts after thawing results relative to the actual number of cells counted just before vialing results in ˜89% Recovery.

RPE purity/identity (CRALBP/PMEL17) was evaluated at batch release. RPE cells at the end of P2 were 96.65% double positive for RPE purity/identity.

### Polarization

Potency assay was performed. Results of net TEER of cells at day 14 in addition to polarized cytokines secretion are presented in Table 40, below.

TABLE 40
Current formal potency assay.

| Net TEER (Ω*cm²)* | PEDF ratio (apical/basal) | VEGF ration (basal/apical) |
|---|---|---|
| 293 | 4.00 | 1.89 |

*per QC-REP-012 and FIG. 1

In addition, frozen samples were thawed and tested in modified potency assay. Results are summarized in Table 41.

TABLE 41
Modified direct potency assay.

| Net TEER (Ω*cm²)* | PEDF ratio (apical/basal) | VEGF ratio (basal/apical) |
|---|---|---|
| 262 | 4.36 | 1.77 |

*per QC-REP-012 and FIG. 1

hESCs Residuals

Cells were thawed and stained for TRA-1-60/Oct-4 in order to determine the presence of residual hESCs. % of hESCs was below limit of detection (0.0004%).

### Karyotype Analysis

Frozen vials at P2 were thawed, cultured for two passages, and fixed for karyotype analysis. Karyotype of cells was normal—3 identical chromosomal depletions or 2 identical additions were not found in the sample.

### Discussion

Process development of OPREGEN® manufacturing towards a commercial process involves FFMD procedure of FF hESCs in addition to culturing RPE cells in large scale system under monitored and controlled conditions. This report summarizes the production process involves FFMD and RPE expansion in 3 L SUB of cells manufactured in CCN GMP facility. The first stage of FFMD process involves the expansion of FF hESCs. Cells pluripotency at each of the hESCs expansion passages was high and over 95% for all tested markers. As expected, a moderate reduction in pluripotency markers expression was observed between hESCs expansion I and hESCs expansion II steps.

Evaluation of RPE purity/identity at the end of differentiation and at the end of P0, demonstrated successful differentiation of cells from the cell bank into 45.96% CRALBP/PMEL17-expressing RPE cells at the end of the differentiation stage. Passaging and culturing of the differentiated cells enriched the RPE population and at the end of P0, co-expression of CRALBP/PMEL17 markers was 97.96%. These results were in the range of RPE purity at these stages as seen in previous FFMD runs. Moreover, the PEDF concentration in culture media was elevated as the differentiation up to P0 of RPE expansion procedure proceeds; from 256.19 ng/ml/day at the end of NIC & Activin A step to 4,687.02 ng/ml/day at the end of P0, affirming the enrichment and maturation of RPE population, for the selected indicative points according to the PEDF selected results.

Ultimately at the end of the process cells were cultured for P2 passage on MC in a semi-automated controlled closed system (Eppendorf's BioFlo 320 console), that monitors the BioBLU 3 L SUB. At the end of P2, cells were harvested and cryopreserved as OPREGEN® TAI. RPE cells produced in this developed FFMD and large scale non-GMP engineering run met all OPREGEN® required acceptance criteria in all batch release tests performed. Taking together, these results qualifying the developed process of OPREGEN® non-GMP engineering run of CCN-FFHESC-01 MCB.

### Conclusion

Non-GMP engineering run—FFMD large scale OPREGEN® production process meet OPREGEN® TAI IPCs and release criteria; no hESCs residuals are present in OPREGEN® TAI, and RPE cells' purity has not been compromised. Furthermore, the cells retain their biological activity and met OPREGEN® acceptance criteria of % Viability and % Recovery.

TABLE 42

| Test | Specification | Test # | Result |
|---|---|---|---|
| Cell Viability ± S.E | ≥70% | REC-018 | 96% ± 0.9% |
| Cell recovery | 75-125% | REC-018 | 78% ± 4.0% |
| Total Cell Number/1 | 75-125% of original | REC-018 | 1.56 × 10⁶ ± 80 × 10³ |
| mL ± S.E | concentration: | | |
| | ≥1.20 × 10⁶ cells/mL for | | |
| | 2M dose | | |
| RPE Purity/Identity: | ≥95.00% | RM-137 | 96.65% |
| % CRALBP + PMEL17 + | | | |
| cells | | | |
| hESC residuals: | <0.01000% | HI-53 | <0.00004% (BLOD) |
| % Oct4 + TRA-1-60 + | | | |
| cells | | | |
| Karyotype | less than 3 identical | Kar-018 | less than 3 identical |
| | deletions or 2 identical | | deletions or 2 |
| | additions | | identical additions |

### Example 5

A method was developed RPE to grow on the Star Plus microcarriers (Solohil), as was established for the fetal derived RPE, for expansion of hESC derived RPE in a large scale closed and controlled environment. In our closed system differentiated RPE cells are inoculated within a single bioreactor containing microcarriers that were screened for optimal RPE yield and quality. Cells oxygen consumption is automatically monitored and controlled as well as monitoring PH metabolites and temperature. Feeding regimen is done in Fed batch mode in which fresh media and glucose are added as required. All manipulation including microcarriers and media addition, cell sampling harvesting and filtration are done in controlled and closed environment by using tube welding of single use bags, until the final product of cell suspension in cryomedium is automatically dispensed into cryovials following controlled large scale freezing of thousands of up to 2300 vials per 1 hour freezing session.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

In the claims, reference to an element in the singular is not intended to mean “one and only one” unless explicitly so stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

1. A method for the expansion of retinal pigment epithelial (RPE) cells, the method comprising:
a) providing a population of RPE cells, wherein the population of RPE cells was differentiated from pluripotent stem cells;
b) inoculating a medium comprising a first suspendable cell support matrix with the population of RPE cells; and
c) expanding the population of RPE cells on the first suspendable cell support matrix in dynamic suspension to provide an expanded population of RPE cells.

2. The method of claim 1, wherein prior to step a) the population of RPE cells was expanded on a solid surface under static conditions.

3. (canceled)

4. The method of claim 1, wherein prior to step a) the population of RPE cells was expanded on a solid surface under dynamic conditions.

5-8. (canceled)

9. The method of claim 1, wherein the first suspendable cell support matrix comprises a microcarrier.

10. The method of claim 1, wherein the population of RPE cells is provided from an intermediate cell bank.

11. The method of claim 1, wherein the first

12. The method of claim 1, wherein differentiation of the population of RPE cells from pluripotent stem cells comprises:
i. expansion of pluripotent stem cells on a solid surface under conditions that maintain pluripotency of the pluripotent stem cells to provide expanded pluripotent stem cells;
ii. differentiating the expanded pluripotent stem cells in a medium comprising a differentiating agent and optionally a growth factor for a period of time to provide the population of RPE cells.

13. (canceled)

14. The method of claim 12, wherein the solid surface comprises a second suspendable cell support matrix and the pluripotent stem cells are expanded in dynamic culture.

15. The method of claim 12, wherein step ii comprises differentiating the expanded pluripotent stem cells on a third suspendable cell support matrix in dynamic culture.

16. The method of claim 15, wherein the expanded pluripotent stem cells from step i remain attached to the second suspendable cell support matrix in step ii.

17-18. (canceled)

19. The method of claim 15, wherein at least two of the first suspendable cell support matrix, second suspendable cell support matrix, and third suspendable cell support matrix are the same.

20. The method of claim 15, wherein the first suspendable cell support matrix, second suspendable cell support matrix, and third suspendable cell support matrix are different.

21. The method of claim 12, wherein step ii comprises differentiating the expanded pluripotent stem cells on a culture plate in static culture.

22. The method of claim 14, wherein the pluripotent stem cells are grown into a monolayer adherent to the second suspendable cell support matrix and/or third suspendable cell support matrix.

23. The method of claim 12, wherein the conditions for maintaining pluripotency are feeder cell free.

24. The method of claim 12, wherein the conditions for maintaining pluripotency comprise a feeder cell population.

25. The method of claim 12, wherein the differentiating reagent is nicotinamide.

26. The method of claim 12, wherein the growth factor is a member of the TGFβ family.

27. The method of claim 15, wherein the first suspendable cell support matrix, second suspendable cell support matrix, and/or third suspendable cell support matrix comprises polystyrene, surface-modified polystyrene, chemically modified polystyrene, cross-linked dextran, cellulose, acrylamide, collagen, alginate, gelatin, glass, DEAE-dextran, or a combination thereof.

28. The method of claim 27, wherein the first microcarrier, second suspendable cell support matrix r, and/or third suspendable cell support matrix is spherical, ellipsoidal, rod-shaped, disc-shaped, porous, non-porous, smooth, flat, or a combination thereof.

29. The method of claim 15, wherein the first suspendable cell support matrix, second suspendable cell support matrix, and/or third suspendable cell support matrix is coated with laminin, Matrigel, collagen, poly-lysine, poly-L-lysine, poly-D-lysine, vitronectin, fibronectin, tenascin, dextran, a peptide, their derivatives, or a combination thereof.

30. The method of claim 2, wherein the solid surface is uncoated or coated with laminin, Matrigel, collagen, poly-lysine, poly-L-lysine, poly-D-lysine, vitronectin, fibronectin, tenascin, dextran, a peptide, their derivatives or a combination thereof.

31. The method of claim 1, wherein the population of RPE cells has a population doubling level between 2-4 during P0, 2-3 during P1, and 1-2 during P2 in step c).

32. The method of claim 1, wherein the population of RPE cells is seeded in the presence of 2%-20% Human serum/DMEM during P0, during P1, and during P2 in step c).

33. The method of claim 1, wherein the population of RPE cells is seeded on solid substrate in dynamic culture during P0, during P1, and during P2 in step c).

34. The method of claim 1, wherein the population of RPE cells was seeded on solid substrate in dynamic culture in cell density of 50,000 cells/cm²-120,000 cells/cm² during P0, during P1, and during P2 in step c).

35. The method of claim 1, wherein the population of RPE cells was seeded in dynamic culture on solid substrate with surface area of 2.5 cm²/ml-10 cm²/ml during P0, during P1, and during P2 in step c).

36. The method of claim 1, wherein step c) comprises maintaining % dissolved oxygen above 30%.

37. The method of claim 1, wherein step c) comprises initial a growth media volume starting at 50% of total system growth chamber volume, and wherein a growth media volume of 16.6% of total system growth chamber volume is added every 2-4 days.

38. The method of claim 1, wherein the expanded population of RPE cells is characteristic of mature RPE cells.

39. The method of claim 1, wherein the mature RPE cells are double positive for cellular retinaldehyde-binding protein (CRALBP) and premelanosome protein (PMEL17) at greater than 95% as measured by flow cytometry.

40. The method of claim 39, wherein the mature RPE cells generate a polarized monolayer post thawing having net transepithelial electrical resistance (TEER) >100Ω*cm² and polarized secretion of PEDF and VEGF.

41. The method of claim 40, wherein the mature RPE cells are cryopreserved and ready for administration to a subject upon thawing.

42. The method of claim 38, wherein the mature RPE cells comprise <0.01% pluripotent stem cells as confirmed by a high accuracy flow cytometry method (FCM), and are negative for TRA-1-60/Oct-4 as measured by flow cytometry.

43. (canceled)

44. The method of claim 1, wherein the dynamic suspension is performed in a single-use bioreactor.

45. (canceled)

46. A method of treating a disorder or disease of the eye, the method comprising transplanting into the retinal tissue of a patient in need thereof a pharmaceutical composition comprising RPE cells generated by the method of claim 1.

47-52. (canceled)

53. A pharmaceutical composition comprising the cells generated by the method of claim 1.

54. (canceled)
